(19) United States
(12) Patent Application Publication
(10) Pub. No.: US 2025/0171531 A1
(43) Pub. Date: May 29, 2025

(54) IL-2 TRAP MOLECULES

(71) Applicants: Orionis Biosciences, Inc., Waltham, MA (US); Orionis Biosciences BV, Zwijnaarde (BE)

(72) Inventors: Nikolai KLEY, Waltham, MA (US); Erik DELPA, Zwijnaarde (BE); Lennart ZABEAU, Zwijnaarde (BE); Jan TAVERNIER, Zwijnaarde (BE)

(21) Appl. No.: 18/837,392

(22) PCT Filed: Feb. 9, 2023
(86) PCT No.: PCT/US2023/062266

§ 371 Date: Aug. 9, 2024

Related U.S. Application Data
(60) Provisional application No. 63/308,572, filed on Feb. 10, 2022.

(51) Int. Cl. C07K 16/24; C07K 16/28

(52) U.S. Cl. CPC C07K 16/246; C07K 16/2815; C07K 2317/31; C07K 2317/569; C07K 2317/622; C07K 2317/76

(57) ABSTRACT

The present disclosure relates to compositions and methods of reducing, mitigating, or eliminating endogenous IL-2 signaling and/or binding to the IL-2 alpha-beta-gamma receptor using bi-functional molecules.

### CROSS-REFERENCE TO RELATED APPLICATIONS

This application claims the benefit of U.S. Provisional Patent Application No. 63/308,572, filed Feb. 10, 2022, the entire contents of which are hereby incorporated by reference in their entirety.

### FIELD

The present disclosure relates, in part, to compositions and methods of reducing, mitigating, or eliminating endogenous IL-2 signaling and/or binding to the IL-2 alpha-beta-gamma receptor.

### DESCRIPTION OF THE TEXT FILE SUBMITTED ELECTRONICALLY

This application contains a Sequence Listing in XML format submitted electronically herewith via Patent Center. The contents of the XML copy, created on Feb. 2, 2023, is named “ORN-085PC_114384-5085.xml” and is 72,095 bytes in size. The Sequence Listing is incorporated herein by reference in its entirety.

### BACKGROUND

Interleukin-2 (IL-2) is well known as a cytokine with potent anti-tumoral activities based on the strong activation of cytotoxic T lymphocytes (CTLs) and natural killer (NKs) cells. In fact, IL-2 was the first immunotherapy ever used (in 1984) and is now FDA approved in metastatic melanoma and metastatic renal cell carcinoma. IL-2 can signal via an intermediate affinity heterodimeric (IL-2Rbeta: gamma common) or a high affinity heterotrimeric (IL-2Ralpha: IL-2Rbeta: gamma common) receptor. Signaling in resting CTLs and NKs occurs via the beta: gamma receptor, while Tregs and other cells that might be responsible for toxicity (e.g. lung epithelial cells) strongly express IL-2Ralpha, and hence use the heterotrimeric receptor for IL-2 signaling. The involvement of IL-2Ralpha chain in the activated receptor complex increases affinity by 100-fold, with a similar effect on biologic activity as a consequence thereof.

The broad use of IL-2 as therapeutic in cancer is hampered, however, by (i) strong activation of regulatory T (Treg) cells which dampen the anti-tumor response, and (ii) toxic side-effects, of which the vascular leakage syndrome (VLS) is best described. Accordingly, approaches to mitigate and/or eliminate the associated toxicity are needed.

### SUMMARY

In one aspect, the present disclosure provides methods of reducing, mitigating, or eliminating endogenous IL-2 signaling and/or binding to the IL-2 alpha-beta-gamma receptor in a subject by administering to the subject a construct comprising (i) an anti-IL-2 antibody, antibody format, or antigen binding portion thereof, which binds IL-2 in a manner that disrupts or blocks IL-2 interaction with the IL-2 alpha-beta-gamma receptor, and (ii) one or more antibody, antibody format, or antigen binding portion thereof directed against an IL-2-responsive cell type marker, wherein the anti-IL-2 antibody, antibody format, or antigen binding portion thereof and the one or more antibody, antibody format, or antigen binding portion thereof directed against an IL-2-responsive cell type marker are linked.

In another aspect, the present disclosure provides methods of redirecting endogenous IL-2 signaling from the IL-2 alpha-beta-gamma receptor to the IL-2 beta-gamma receptor on an antigen-expressing cell in a subject by administering to the subject a construct comprising: (i) an anti-IL-2 antibody, antibody format, or antigen binding portion thereof, which binds IL-2 in a manner that disrupts or blocks IL-2 interaction with the IL-2 alpha-beta-gamma receptor, and (ii) one or more antibody, antibody format, or antigen binding portion thereof directed against an IL-2-responsive cell type marker, wherein the anti-IL-2 antibody, antibody format, or antigen binding portion thereof and the one or more antibody, antibody format, or antigen binding portion thereof directed against an IL-2-responsive cell type marker are linked.

In some embodiments, the IL-2-responsive cell type is selected from a cytotoxic T lymphocyte (CTL), a natural killer cell (NK), a Thelp cell, and a gamma-delta T cell. In such embodiments, the CTL or NK cell or Thelp cell or gamma-delta T cell marker is selected from one or more of CD3, CD4, CD8, CD28, CD45, CD54, CD56, CD94, CD95, CD226, NKG2D, NKp30, NKp44, NKp46, TCRab, carbonic anhydrase IX (CAIX), 5T4, CD19, CD20, CD22, CD30, CD33, CD38, CD47, CS1, CD138, Lewis-Y, L1-CAM, MUC16, ROR-1, IL13Ralpha2, gp100, prostate stem cell antigen (PSCA), prostate-specific membrane antigen (PSMA), B-cell maturation antigen (BCMA), human papillomavirus type 16 E6 (HPV-16 E6), CD171, folate receptor alpha (FR-alpha), GD2, human epidermal growth factor receptor 2 (HER2), mesothelin, EGFRvlll, fibroblast activation protein (FAP), carcinoembryonic antigen (CEA), vascular endothelial growth factor receptor 2 (VEGF-R2), E-Cadherin, CCR5, CCR6, CD27/TNFRSF7, CD27 Ligand/TNFSF7, CD40/TNFRSF5, CD40 Ligand/TNFSF5, CD83, CD161, CD161/NK1.1, CXC4, Dectin-1/CLEC7A, Fas/TNFRSF6/CD95, Fas Ligand/TNFSF6, Fc gamma RIII (CD16), Fc gamma RIIIA/CD16a, Fc gamma RIIIB/CD16b, ICOS, IL-18 R alpha/IL-1 R5, IL-23R, NKG2D/CD314, NKG2E, Occludin, TCR gamma/delta, TLR2, and TRAIL/TNFSF10.

In various embodiments, endogenous IL-2 is redirected from Treg cells to CTLs and/or NK cells and/or Thelp cells and/or gamma-delta T cells. In such embodiments, the method reduces IL-2 signaling on a Treg cell. In certain embodiments, the method results in neutralization of IL-2 binding and/or interaction with the IL-2 alpha-beta-gamma receptor. In various embodiments, the method causes selective proliferation and/or survival of CTLs or NK cells or Thelp cells.

In some embodiments, the method reduces the ratio of IL-2Ralpha-beta-gamma-mediated signaling to IL-2Rbeta-gamma-mediated signaling. In embodiments, the method reduces the ratio of IL-2-modulated Treg cells to IL-2-modulated CTLs or NKs or Thelp cells. In some embodiments, the method decreases IL-2 mediated STAT signaling on Treg cells. In embodiments, the method increases IL-2-mediated STAT signaling on CTLs or NKs or Thelp cells.

In some embodiments, the method increases IL-2 sensitivity on CTLs or NKs or Thelp cells, and the method decreases IL-2 sensitivity on Treg cells. In certain embodiments, the method causes the ratio of IL-2 sensitivity on Treg cells to CTLs and/or NK cells and/or Thelp cells to be about 1.

In various embodiments, the method increases anti-tumor activity of IL-2 and decreases activation of Treg cells. In such embodiments, the decrease of activation of Treg cells results in a decrease of T-cell suppression. In some embodiments, the construct does not comprise an exogenous wild-type IL-2 molecule.

In embodiments, the construct further comprises a targeting moiety comprising a recognition domain which specifically binds to a tissue-specific marker. In such embodiments, the tissue-specific marker is selected from endothelial cell surface markers such as ACE, CD14, CD34, CDH5, ENG, ICAM2, MCAM, NOS3, PECAMI, PROCR, SELE, SELP, TEK, THBD, VCAMI, TEM1, TEM8, Clec14A, VWF; smooth muscle cell surface markers such as ACTA2, MYHIO, MYHI 1, MYH9, MYOCD; fibroblast (stromal) cell surface markers such as FAP, ALCAM, CD34, COLIAI, COL1A2, COL3A1, PH-4; epithelial cell surface markers such as CDID, K6IRS2, KRTIO, KRT13, KRT17, KRT18, KRT19, KRT4, KRT5, KRT8, MUCI, TACSTDI; neovasculature markers such as CD13, TFNA, Alpha-v beta-3 (aVB3), E-selectin; and adipocyte surface markers such as ADIPOQ, FABP4, and RETN. In some embodiments, the tissue is the extracellular matrix (ECM). In such embodiments, the ECM marker is selected from a tenascin, optionally selected from tenascin-C, tenascin-R, tenascin-X, and tenascin-W, a fibronectin, a fibrin, a laminin, and a nidogen/entactin. In particular embodiments, the tenascin is tenascin-CA1.

In some embodiments, the construct of the present invention acts synergistically when used in combination with Chimeric Antigen Receptor (CAR) T-cell therapy. In an illustrative embodiment, the construct acts synergistically when used in combination with CAR T-cell therapy in treating tumor or cancer. In an embodiment, the construct acts synergistically when used in combination with CAR T-cell therapy in treating blood-based tumors. In an embodiment, the construct acts synergistically when used in combination with CAR T-cell therapy in treating solid tumors. For example, use of the construct and CAR T-cells may act synergistically to reduce or eliminate the tumor or cancer, or slow the growth and/or progression and/or metastasis of the tumor or cancer. In various embodiments, the construct of the invention induces CAR T-cell division. In various embodiments, the construct of the invention induces CAR T-cell proliferation. In various embodiments, the construct of the invention prevents anergy of the CAR T-cells.

In various embodiments, the CAR T-cell therapy comprises CAR T-cells that target antigens (e.g., tumor antigens) such as, but not limited to, carbonic anhydrase IX (CAIX), 5T4, CD19, CD20, CD22, CD30, CD33, CD38, CD47, CS1, CD138, Lewis-Y, L1-CAM, MUC16, ROR-1, IL13Ralpha2, gp100, prostate stem cell antigen (PSCA), prostate-specific membrane antigen (PSMA), B-cell maturation antigen (BCMA), human papillomavirus type 16 E6 (HPV-16 E6), CD171, folate receptor alpha (FR-alpha), GD2, human epidermal growth factor receptor 2 (HER2), mesothelin, EGFRvlll, fibroblast activation protein (FAP), carcinoembryonic antigen (CEA), and vascular endothelial growth factor receptor 2 (VEGF-R2), as well as other tumor antigens well known in the art. Additional illustrative tumor antigens include, but are not limited to MART-1/Melan-A, gp100, Dipeptidyl peptidase IV (DPPIV), adenosine deaminase-binding protein (ADAbp), cyclophilin b, Colorectal associated antigen (CRC)-0017-1A/GA733, Carcinoembryonic Antigen (CEA) and its immunogenic epitopes CAP-1 and CAP-2, etv6, aml1, Prostate Specific Antigen (PSA) and its immunogenic epitopes PSA-1, PSA-2, and PSA-3, T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, alpha-fetoprotein, E-cadherin, alpha-catenin, beta-catenin and gamma-catenin, p120ctn, gp100 Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, NA, EBV-encoded nuclear antigen (EBNA)-1, brain glycogen phosphorylase, SSX-1, SSX-2 (HOM-MEL-40), SSX-1, SSX-4, SSX-5, SCP-1 CT-7, c-erbB-2, CD19, CD37, CD56, CD70, CD74, CD138, AGS16, MUC1, GPNMB, Ep-CAM, PD-L1, and PD-L2.

Illustrative CAR T-cell therapies include, but are not limited to, JCAR014 (Juno Therapeutics), JCAR015 (Juno Therapeutics), JCAR017 (Juno Therapeutics), JCAR018 (Juno Therapeutics), JCAR020 (Juno Therapeutics), JCAR023 (Juno Therapeutics), JCAR024 (Juno Therapeutics), CTL019 (Novartis), KTE-C19 (Kite Pharma), BPX-401 (Bellicum Pharmaceuticals), BPX-501 (Bellicum Pharmaceuticals), BPX-601 (Bellicum Pharmaceuticals), bb2121 (Bluebird Bio), CD-19 Sleeping Beauty cells (Ziopharm Oncology), UCART19 (Cellectis), UCART123 (Cellectis), UCART38 (Cellectis), UCARTCS1 (Cellectis), OXB-302 (Oxford BioMedica, MB-101 (Mustang Bio) and CAR T-cells developed by Innovative Cellular Therapeutics.

In various embodiments, the subject is afflicted with cancer.

In another aspect, the present disclosure provides for methods of reducing, mitigating, or eliminating endogenous IL-2 signaling and/or binding to the IL-2 alpha-beta-gamma receptor in a subject. In another aspect, the present disclosure provides for methods of redirecting endogenous IL-2 signaling from the IL-2 alpha-beta-gamma receptor to the IL-2 beta-gamma receptor on an antigen-expressing cell in a subject. In such aspects, the methods comprise administering to the subject a construct comprising (i) an anti-IL-2 antibody, antibody format, or antigen binding portion thereof, which binds IL-2 in a manner that disrupts or blocks IL-2 interaction with the IL-2 alpha-beta-gamma receptor, and (ii) one or more antibody, antibody format, or antigen binding portion thereof directed against a Treg cell marker wherein the anti-IL-2 antibody, antibody format, or antigen binding portion thereof and the one or more antibody, antibody format, or antigen binding portion thereof directed against a Treg cell marker are linked.

In some embodiments, the endogenous IL-2 is redirected from activated CTL or NK or Thelp cells to Treg cells. In embodiments, the Treg cell marker is CTLA-4, CD25, GITR, CD127, LAG-3, PD-1, and CCR8.

In some embodiments, the antibody, antibody format, or antigen binding portion thereof directed against a Treg cell marker is an anti-CTLA-4, anti-CD25, anti-GITR, anti-CD127, or anti-LAG-3 antibody, antibody format, or antigen binding portion thereof. In embodiments, the anti-CTLA-4, anti-CD25, anti-GITR, anti-CD127, or anti-LAG-3 antibody format is a single-domain antibody scFv, a recombinant heavy-chain-only antibody (VHH), a single-chain antibody (scFv), a shark heavy-chain-only antibody (VNAR), a Fv, a Fab, a Fab′, or a F(ab′)₂. In particular embodiments, the anti-CTLA-4, anti-CD25, anti-GITR, anti-CD127, or anti-LAG-3 antibody format is a VHH or scFv.

In some embodiments, the method results in neutralization of IL-2 binding and/or interaction with the IL-2 alpha-beta-gamma receptor. In embodiments, the method reduces IL-2 signaling on an activated CTL or NK or Thelp cell. In some embodiments, the method causes selective proliferation and/or survival of Treg cells.

In embodiments, the method reduces the ratio of IL-2Ralpha-beta-gamma-mediated signaling to IL-2Rbeta-gamma-mediated signaling. In some embodiments, the method reduces the ratio of IL-2-modulated CTL cells to IL-2-modulated Treg cells.

In embodiments, the construct further comprises a targeting moiety comprising a recognition domain which specifically binds to a tissue-specific marker. In such embodiments, the tissue-specific marker is selected from endothelial cell surface markers such as ACE, CD14, CD34, CDH5, ENG, ICAM2, MCAM, NOS3, PECAMI, PROCR, SELE, SELP, TEK, THBD, VCAMI, TEM1, TEM8, Clec14A, VWF; smooth muscle cell surface markers such as ACTA2, MYHIO, MYHI 1, MYH9, MYOCD; fibroblast (stromal) cell surface markers such as FAP, ALCAM, CD34, COLIAI, COL1A2, COL3A1, PH-4; epithelial cell surface markers such as CDID, K6IRS2, KRTIO, KRT13, KRT17, KRT18, KRT19, KRT4, KRT5, KRT8, MUCI, TACSTDI; neovasculature markers such as CD13, TFNA, Alpha-v beta-3 (aVB3), E-selectin; and adipocyte surface markers such as ADIPOQ, FABP4, and RETN. In some embodiments, the tissue is the extracellular matrix (ECM). In such embodiments, the ECM marker is selected from a tenascin, optionally selected from tenascin-C, tenascin-R, tenascin-X, and tenascin-W, a fibronectin, a fibrin, a laminin, and a nidogen/entactin. In particular embodiments, the tenascin is tenascin-CA1.

In various embodiments, the subject is afflicted with cancer or an auto-immune disease or disorder.

### BRIEF DESCRIPTION OF THE DRAWINGS

FIG. 1 depicts a schematic representing a non-limiting anti-IL-2 construct mechanism. The top panel shows that the involvement of IL-2Ralpha chain in the activated receptor complex increases affinity by 100-fold, with a similar effect on biologic activity as a consequence thereof. The middle panel shows that a neutralizing anti-IL-2 scFv (a non-limiting, illustrative embodiment) can decrease the involvement of the IL-2Ralpha chain in the activated receptor complex on a Treg cell. The bottom panel shows that the use of a neutralizing anti-IL-2 scFv linked to an anti-CD8 scFv increases the IL-2 signaling on activated CTL cells.

FIG. 2 shows binding of anti-IL-2 constructs to IL-2 in bio-layer interferometry. In this figure, the illustrative anti-IL-2 antibody, antibody format, or antigen binding portion thereof, is labelled as NARA1 or TCB2. In this figure, the illustrative antibody, antibody format, or antigen binding portion thereof, directed against an IL-2-responsive cell type marker is labelled as OKT8, which is a CD8a monoclonal antibody.

FIG. 3 shows binding of anti-IL-2 constructs to CD8 alpha in bio-layer interferometry. In this figure, the illustrative anti-IL-2 antibody, antibody format, or antigen binding portion thereof, is labelled as NARA1 or TCB2. In this figure, the illustrative antibody, antibody format, or antigen binding portion thereof, directed against an IL-2-responsive cell type marker is labelled as OKT8, which is a CD8a monoclonal antibody.

FIG. 4 depicts the effect of anti-IL-2 constructs on the binding of IL-2 to the IL-2Ralpha in bio-layer interferometry. In this figure, the illustrative anti-IL-2 antibody, antibody format, or antigen binding portion thereof, is labelled as NARA1 or TCB2. In this figure, the illustrative antibody, antibody format, or antigen binding portion thereof, directed against an IL-2-responsive cell type marker is labelled as OKT8, which is a CD8a monoclonal antibody.

FIG. 5 shows the effect of anti-IL-2 constructs on STAT5 phosphorylation in CD8+ and Treg cells. In this figure, the illustrative anti-IL-2 antibody, antibody format, or antigen binding portion thereof, is labelled as NARA1 or TCB2. In this figure, the illustrative antibody, antibody format, or antigen binding portion thereof, directed against an IL-2-responsive cell type marker is labelled as OKT8, which is a CD8a monoclonal antibody.

FIG. 6 shows the effect of anti-IL-2 construct in Fc-format on STAT5 phosphorylation in CD8+ and Treg cells. In this figure, the illustrative anti-IL-2 antibody, antibody format, or antigen binding portion thereof, is labelled as NARA1 or TCB2. In this figure, the illustrative antibody, antibody format, or antigen binding portion thereof, directed against an IL-2-responsive cell type marker is labelled as OKT8, which is a CD8a monoclonal antibody.

FIG. 7 depicts the evaluation of a CD8 VHH as targeting domain in the anti-IL-2 construct. In this figure, the illustrative anti-IL-2 antibody, antibody format, or antigen binding portion thereof, is labelled as TCB2.

### DETAILED DESCRIPTION

The present technology is based, in part, on the discovery of various mechanisms to increase the anti-tumor activity of IL-2, while reducing the activation of Tregs and other cells involved in toxicity. Such mechanisms that can achieve this include: (i) eliminating the involvement and/or recruitment of the IL-2Ralpha chain in the activated receptor (e.g., by mutation of the binding site, site-specific PEGylation, shielding of the binding-site using an antibody or receptor fragments), and/or (ii) targeting IL-2 to desired cells (CTLs and NKs) by antibody or fragments thereof.

In one aspect, the present disclosure provides methods of reducing, mitigating, or eliminating endogenous IL-2 signaling and/or binding to the IL-2 alpha-beta-gamma receptor in a subject, the methods comprising administering to the subject a construct comprising (i) an anti-IL-2 antibody, antibody format, or antigen binding portion thereof, which binds IL-2 in a manner that disrupts or blocks IL-2 interaction with the IL-2 alpha-beta-gamma receptor, and (ii) one or more antibody, antibody format, or antigen binding portion thereof directed against an IL-2-responsive cell type marker, wherein the anti-IL-2 antibody, antibody format, or antigen binding portion thereof and the one or more antibody, antibody format, or antigen binding portion thereof directed against an IL-2-responsive cell type marker are linked.

In an aspect, the present disclosure provides methods of reducing, mitigating, or eliminating endogenous IL-2 signaling and/or binding to the IL-2 alpha-beta-gamma receptor in a subject, the methods comprising contacting a cell with a construct comprising (i) an anti-IL-2 antibody, antibody format, or antigen binding portion thereof, which binds IL-2 in a manner that disrupts or blocks IL-2 interaction with the IL-2 alpha-beta-gamma receptor, and (ii) one or more antibody, antibody format, or antigen binding portion thereof directed against an IL-2-responsive cell type marker, wherein the anti-IL-2 antibody, antibody format, or antigen binding portion thereof and the one or more antibody, antibody format, or antigen binding portion thereof directed against an IL-2-responsive cell type marker are linked. In such embodiments, the cell is administered to the subject. In embodiments, the cell is autologous or allogenic.

In another aspect, the present disclosure provides methods of redirecting endogenous IL-2 signaling from the IL-2 alpha-beta-gamma receptor to the IL-2 beta-gamma receptor on an antigen-expressing cell in a subject, the methods comprising administering to the subject a construct comprising (i) an anti-IL-2 antibody, antibody format, or antigen binding portion thereof, which binds IL-2 in a manner that disrupts or blocks IL-2 interaction with the IL-2 alpha-beta-gamma receptor, and (ii) one or more antibody, antibody format, or antigen binding portion thereof directed against an IL-2-responsive cell type marker, wherein the anti-IL-2 antibody, antibody format, or antigen binding portion thereof and the one or more antibody, antibody format, or antigen binding portion thereof directed against an IL-2-responsive cell type marker are linked.

In an aspect, the present disclosure provides methods of redirecting endogenous IL-2 signaling from the IL-2 alpha-beta-gamma receptor to the IL-2 beta-gamma receptor on an antigen-expressing cell in a subject, the methods comprising contacting a cell with a construct comprising (i) an anti-IL-2 antibody, antibody format, or antigen binding portion thereof, which binds IL-2 in a manner that disrupts or blocks IL-2 interaction with the IL-2 alpha-beta-gamma receptor, and (ii) one or more antibody, antibody format, or antigen binding portion thereof directed against an IL-2-responsive cell type marker, wherein the anti-IL-2 antibody, antibody format, or antigen binding portion thereof and the one or more antibody, antibody format, or antigen binding portion thereof directed against an IL-2-responsive cell type marker are linked. In such embodiments, the cell is administered to the subject. In embodiments, the cell is autologous or allogenic.

In another aspect, the present disclosure provides for methods of reducing, mitigating, or eliminating endogenous IL-2 signaling and/or binding to the IL-2 alpha-beta-gamma receptor in a subject, the methods comprising administering to the subject a construct comprising (i) an anti-IL-2 antibody, antibody format, or antigen binding portion thereof, which binds IL-2 in a manner that disrupts or blocks IL-2 interaction with the IL-2 alpha-beta-gamma receptor, and (ii) one or more antibody, antibody format, or antigen binding portion thereof directed against a Treg cell marker wherein the anti-IL-2 antibody, antibody format, or antigen binding portion thereof and the one or more antibody, antibody format, or antigen binding portion thereof directed against a Treg cell marker are linked.

In an aspect, the present disclosure provides for methods of reducing, mitigating, or eliminating endogenous IL-2 signaling and/or binding to the IL-2 alpha-beta-gamma receptor in a subject, the methods comprising contacting a cell with a construct comprising (i) an anti-IL-2 antibody, antibody format, or antigen binding portion thereof, which binds IL-2 in a manner that disrupts or blocks IL-2 interaction with the IL-2 alpha-beta-gamma receptor, and (ii) one or more antibody, antibody format, or antigen binding portion thereof directed against a Treg cell marker wherein the anti-IL-2 antibody, antibody format, or antigen binding portion thereof and the one or more antibody, antibody format, or antigen binding portion thereof directed against a Treg cell marker are linked. In such embodiments, the cell is administered to the subject. In embodiments, the cell is autologous or allogenic.

In another aspect, the present disclosure provides for methods of redirecting endogenous IL-2 signaling from the IL-2 alpha-beta-gamma receptor to the IL-2 beta-gamma receptor on an antigen-expressing cell in a subject, the methods comprising administering to the subject a construct comprising (i) an anti-IL-2 antibody, antibody format, or antigen binding portion thereof, which binds IL-2 in a manner that disrupts or blocks IL-2 interaction with the IL-2 alpha-beta-gamma receptor, and (ii) one or more antibody, antibody format, or antigen binding portion thereof directed against a Treg cell marker wherein the anti-IL-2 antibody, antibody format, or antigen binding portion thereof and the one or more antibody, antibody format, or antigen binding portion thereof directed against a Treg cell marker are linked.

In another aspect, the present disclosure provides for methods of redirecting endogenous IL-2 signaling from the IL-2 alpha-beta-gamma receptor to the IL-2 beta-gamma receptor on an antigen-expressing cell in a subject, the methods comprising contacting a cell with a construct comprising (i) an anti-IL-2 antibody, antibody format, or antigen binding portion thereof, which binds IL-2 in a manner that disrupts or blocks IL-2 interaction with the IL-2 alpha-beta-gamma receptor, and (ii) one or more antibody, antibody format, or antigen binding portion thereof directed against a Treg cell marker wherein the anti-IL-2 antibody, antibody format, or antigen binding portion thereof and the one or more antibody, antibody format, or antigen binding portion thereof directed against a Treg cell marker are linked. In such embodiments, the cell is administered to the subject. In embodiments, the cell is autologous or allogenic.

### Anti-IL-2 Construct

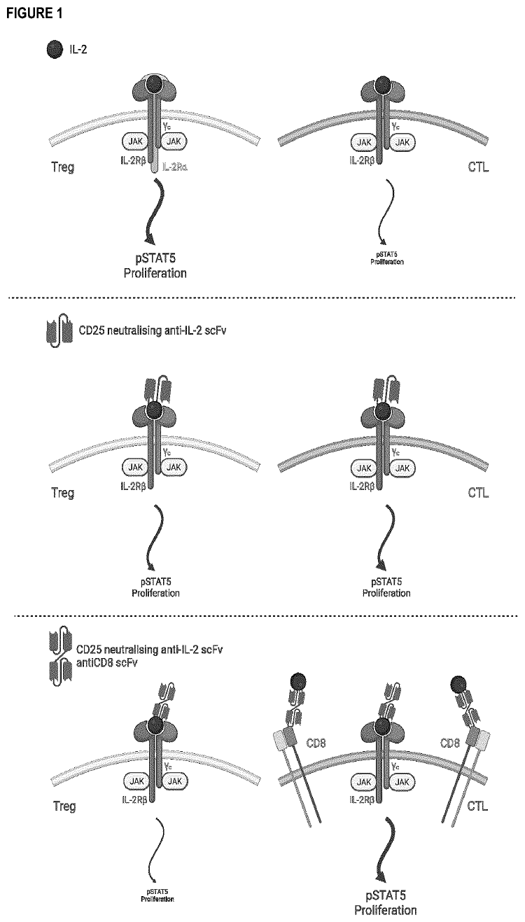

IL-2 can signal via an intermediate affinity heterodimeric (IL-2Rbeta: gamma common) or a high affinity heterotrimeric (IL-2Ralpha: IL-2Rbeta: gamma common) receptor. Signaling in resting CTLs and NKs occurs via the beta: gamma receptor, while Tregs and other cells that might be responsible for toxicity (e.g. lung epithelial cells) strongly express IL-2Ralpha, and hence use the heterotrimeric receptor for IL-2 signaling. The involvement of IL-2Ralpha chain in the activated receptor complex increases affinity by 100-fold, with a similar effect on biologic activity as a consequence thereof (see FIG. 1, upper panel).

In some embodiments, the construct comprises (i) an anti-IL-2 antibody, antibody format, or antigen binding portion thereof, which binds IL-2 in a manner that disrupts or blocks IL-2 interaction with the IL-2 alpha-beta-gamma receptor, and (ii) one or more antibody, antibody format, or antigen binding portion thereof directed against an IL-2-responsive cell type marker. In various embodiments, the construct comprises one or more antibody, antibody format, or antigen binding portion thereof directed against an IL-2-responsive cell type marker. In embodiments, the IL-2-responsive cell type is selected from a cytotoxic T lymphocyte (CTL), a natural killer cell (NK), a Thelp cell, and a gamma-delta T cell. In such embodiments, the CTL or NK cell or Thelp cell or gamma-delta T cell marker is selected from one or more of CD3, CD4, CD8, CD28, CD45, CD54, CD56, CD94, CD95, CD226, NKG2D, NKp30, NKp44, NKp46, TCRab, carbonic anhydrase IX (CAIX), 5T4, CD19, CD20, CD22, CD30, CD33, CD38, CD47, CS1, CD138, Lewis-Y, L1-CAM, MUC16, ROR-1, IL13Ralpha2, gp100, prostate stem cell antigen (PSCA), prostate-specific membrane antigen (PSMA), B-cell maturation antigen (BCMA), human papillomavirus type 16 E6 (HPV-16 E6), CD171, folate receptor alpha (FR-alpha), GD2, human epidermal growth factor receptor 2 (HER2), mesothelin, EGFRvIII, fibroblast activation protein (FAP), carcinoembryonic antigen (CEA), vascular endothelial growth factor receptor 2 (VEGF-R2), E-Cadherin, CCR5, CCR6, CD27/TNFRSF7, CD27 Ligand/TNFSF7, CD40/TNFRSF5, CD40 Ligand/TNFSF5, CD83, CD161, CD161/NK1.1, CXC4, Dectin-1/CLEC7A, Fas/TNFRSF6/CD95, Fas Ligand/TNFSF6, Fc gamma RIII (CD16), Fc gamma RIIIA/CD16a, Fc gamma RIIIB/CD16b, ICOS, IL-18 R alpha/IL-1 R5, IL-23R, NKG2D/CD314, NKG2E, Occludin, TCR gamma/delta, TLR2, and TRAIL/TNFSF10.

In other embodiments, the construct comprises (i) an anti-IL-2 antibody, antibody format, or antigen binding portion thereof, which binds IL-2 in a manner that disrupts or blocks IL-2 interaction with the IL-2 alpha-beta-gamma receptor, and (ii) one or more antibody, antibody format, or antigen binding portion thereof directed against a Treg cell marker. In embodiments, the Treg cell marker is selected from one or more of CTLA-4, CD25, GITR, CD127, LAG-3, PD-1, and CCR8.

In some embodiments, the antibody, antibody format, or antigen binding portion thereof directed against an IL-2-responsive cell type marker, optionally a cytotoxic T lymphocyte (CTL) or natural killer cell (NK) or Thelp cell marker or gamma-delta T cell, is an anti-CD8 antibody, antibody format, or antigen binding portion thereof. In such embodiments, the anti-CD8 antibody format is a single-domain antibody, a recombinant heavy-chain-only antibody (VHH), a single-chain antibody (scFv), a shark heavy-chain-only antibody (VNAR), a Fv, a Fab, a Fab′, or a F(ab′)₂. In particular embodiments, the anti-CD8 antibody format is a VHH or scFv. In certain embodiments, the construct comprises a polypeptide having at least about 90%, at least about 93%, at least about 94%, at least about 95%, at least about 96%, at least about 97%, at least about 98%, or at least about 99% amino acid sequence similarity to one of SEQ ID NO: 1-12. In certain embodiments, the construct comprises a polypeptide having at least about 90%, at least about 93%, at least about 94%, at least about 95%, at least about 96%, at least about 97%, at least about 98%, or at least about 99% amino acid sequence similarity to one of SEQ ID NO: 1-12, without a leader sequence and/or His₆ tag. In embodiments, the construct comprises a polypeptide having the amino acid sequence of one of SEQ ID NO: 1-12. In embodiments, the construct comprises a polypeptide having the amino acid sequence of one of SEQ ID NO: 1-12, without a leader sequence and/or His₆ tag. In embodiments, the construct comprises a polypeptide not having a leader sequence. In embodiments, the construct comprises a polypeptide not having a His₆ tag.

In embodiments, the construct comprises an anti-IL-2 antibody, antibody format, or antigen binding portion thereof that binds IL-2 in a manner that disrupts or blocks IL-2 interaction with the IL-2 alpha-beta-gamma receptor. In such embodiments, the antibody format is a single-chain antibody (scFv), a single-domain antibody, a recombinant heavy-chain-only antibody (VHH), a shark heavy-chain-only antibody (VNAR), a Fv, a Fab, a Fab′, or a F(ab′)₂. In particular embodiments, the antibody, antibody format, or antigen binding portion thereof is a scFv, or variant thereof. In some embodiments, the scFv, or variant thereof, comprises a VL-VH or VH-VL orientation.

In embodiments, the construct does not comprise an exogenous wild-type IL-2 molecule.

In embodiments, the construct comprises NARA1. NARA1 is a monoclonal antibody to human IL-2 that acts like a high-affinity CD25 mimic, thus minimizing association of IL-2 with CD25. See Arenas-Ramirez et al., “Improved cancer immunotherapy by a CD25-mimobody conferring selectivity to human interleukin-2,” Sci Transl Med 8 (367), 2016, the entire contents of which are incorporated by reference.

In embodiments, the construct comprises TCB2. TCB2 is an anti-human IL-2 (hIL-2) monoclonal antibody that selectively stimulates CD8 T and NK cells without overtly activating Tregs. See Lee et al., “TCB2, a new anti-human interleukin-2 antibody, facilitates heterodimeric IL-2 receptor signaling and improves anti-tumor immunity,” Oncoimmunology 9 (1): 1681869, 2019, the entire contents of which are incorporated by reference.

In some embodiments, the construct further comprises a Fc domain. In such embodiments, the construct has an increased half-life as compared to a construct without a Fc domain. In embodiments, the Fc domain is a human IgG1 Fc backbone. In some embodiments, the Fc domain comprises effector mutations selected from one or more of L234A, L235A, P331G, and K322Q, or mutations corresponding thereto. In further embodiments, the Fc domain comprises mutations selected from one or more of S354C and T366W, or mutations corresponding thereto. In additional embodiments, the Fc domain further comprises mutations selected from one or more of Y349C, T366S, L368A, and Y407V, or mutations corresponding thereto.

In some embodiments, the Fc-based construct optionally comprise one or more flexible linkers. In some embodiments, the linker is a single amino acid or a plurality of amino acids that does not affect or reduce the stability, orientation, binding, neutralization, and/or clearance characteristics of the binding regions and the binding protein. In various embodiments, the linker is selected from a peptide, a protein, a sugar, or a nucleic acid. The invention contemplates the use of a variety of linker sequences. In various embodiments, the linker may be derived from naturally-occurring multi-domain proteins or are empirical linkers as described, for example, in Chichili et al., (2013), Protein Sci. 22 (2): 153-167, Chen et al., (2013), Adv Drug Deliv Rev. 65 (10): 1357-1369, the entire contents of which are hereby incorporated by reference. In some embodiments, the linker may be designed using linker designing databases and computer programs such as those described in Chen et al., (2013), Adv Drug Deliv Rev. 65 (10): 1357-1369 and Crasto et al., (2000), Protein Eng. 13 (5): 309-312, the entire contents of which are hereby incorporated by reference. In various embodiments, the linker may be functional. For example, without limitation, the linker may function to improve the folding and/or stability, improve the expression, improve the pharmacokinetics, and/or improve the bioactivity of the present constructs.

In some embodiments, the linker is a polypeptide. In some embodiments, the linker is less than about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is a polypeptide. In some embodiments, the linker is greater than about 100 amino acids long. For example, the linker may be greater than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In some embodiments, the linker is flexible. In another embodiment, the linker is rigid.

In various embodiments, the linker is substantially comprised of glycine and serine residues (e.g. about 30%, or about 40%, or about 50%, or about 60%, or about 70%, or about 80%, or about 90%, or about 95%, or about 97% glycines and serines). For example, in some embodiments, the linker is (Gly₄Ser)ₙ, where n is from about 1 to about 8, e.g. 1, 2, 3, 4, 5, 6, 7, or 8 (SEQ ID NO: 13-SEQ ID NO: 20, respectively). For example, in some embodiments, the linker is (Gly₃Ser)ₙ, where n is from about 1 to about 8, e.g. 1, 2, 3, 4, 5, 6, 7, or 8 (SEQ ID NO: 21-SEQ ID NO: 28, respectively). In an embodiment, the linker sequence is GGSGGSGGGGSGGGGS (SEQ ID NO: 29). Additional illustrative linkers include, but are not limited to, linkers having the sequence GS, LE, GGGGS (SEQ ID NO: 30), (GGGGS)ₙ (n=1-4) (SEQ ID NO: 31-SEQ ID NO: 34). (Gly)₈ (SEQ ID NO: 35), (Gly)₆ (SEQ ID NO: 36), (EAAAK)ₙ (n=1-3) (SEQ ID NO: 37-SEQ ID NO: 39), A (EAAAK)ₙA (n=2-5) (SEQ ID NO: 40-SEQ ID NO: 43), AEAAAKEAAAKA (SEQ ID NO: 44), A (EAAAK)₄ALEA (EAAAK)₄A (SEQ ID NO: 45), PAPAP (SEQ ID NO: 46), KESGSVSSEQLAQFRSLD (SEQ ID NO: 47), EGKSSGSGSESKST (SEQ ID NO: 48), GSAGSAAGSGEF (SEQ ID NO: 49), and (XP)ₙ, with X designating any amino acid, e.g., Ala, Lys, or Glu. In various embodiments, the linker is GGS. In some embodiments, the linker is one or more of GGGSE (SEQ ID NO: 50), GSESG (SEQ ID NO: 51), GSEGS (SEQ ID NO: 52), GEGGSGEGSSGEGSSSEGGGSEGGGSEGGGSEGGS (SEQ ID NO: 53), and a linker of randomly placed G, S, and E every 4 amino acid intervals.

In some embodiments, the linker is a hinge region of an antibody (e.g., of IgG, IgA, IgD, and IgE, inclusive of subclasses (e.g. IgG1, IgG2, IgG3, and IgG4, and IgA1 and IgA2)). In various embodiments, the linker is a hinge region of an antibody (e.g., of IgG, IgA, IgD, and IgE, inclusive of subclasses (e.g. IgG1, IgG2, IgG3, and IgG4, and IgA1 and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2.

In additional embodiments, the construct further comprises a targeting moiety comprising a recognition domain which specifically binds to a tissue-specific marker. For example, the tissue-specific marker can be selected from endothelial cell surface markers such as ACE, CD14, CD34, CDH5, ENG, ICAM2, MCAM, NOS3, PECAMI, PROCR, SELE, SELP, TEK, THBD, VCAMI, TEM1, TEM8, Clec14A, VWF; smooth muscle cell surface markers such as ACTA2, MYHIO, MYHI 1, MYH9, MYOCD; fibroblast (stromal) cell surface markers such as FAP, ALCAM, CD34, COLIAI, COL1A2, COL3A1, PH-4; epithelial cell surface markers such as CDID, K6IRS2, KRTIO, KRT13, KRT17, KRT18, KRT19, KRT4, KRT5, KRT8, MUCI, TACSTDI; neovasculature markers such as CD13, TFNA, Alpha-v beta-3 (αVβ3), E-selectin; and adipocyte surface markers such as ADIPOQ, FABP4, and RETN. In particular embodiments, the tissue is the extracellular matrix (ECM). For example, in embodiments, the ECM marker is selected from a tenascin. In such embodiments, the tenascin is selected from tenascin-C, tenascin-R, tenascin-X, and tenascin-W, a fibronectin, a fibrin, a laminin, and a nidogen/entactin. In particular embodiments, the tenascin is tenascin-CA1.

In various embodiments, the construct is a polypeptide.

In some embodiments, the construct is a nucleic acid. In embodiments, the nucleic acid is or comprises deoxyribonucleic acid (DNA) and/or ribonucleic acid (RNA). In embodiments, the nucleic acid is DNA, optionally selected from linear DNA, DNA fragments, or DNA plasmids. In embodiments, the nucleic acid is an mRNA, optionally comprising one or more non-canonical nucleotides, optionally selected from pseudouridine and 5-methoxyuridine. In particular embodiments, the RNA is modified messenger RNA (mmRNA).

IL-2 Redirection from Treg Cells to an IL-2-Responsive Cell Type

In some embodiments, the construct comprises (i) an anti-IL-2 antibody, antibody format, or antigen binding portion thereof, which binds IL-2 in a manner that disrupts or blocks IL-2 interaction with the IL-2 alpha-beta-gamma receptor, and (ii) one or more antibody, antibody format, or antigen binding portion thereof directed against an IL-2-responsive cell type marker.

In embodiments, the construct comprises an anti-IL-2 antibody, antibody format, or antigen binding portion thereof that binds IL-2 in a manner that disrupts or blocks IL-2 interaction with the IL-2 alpha-beta-gamma receptor. In such embodiments, the antibody format is a single-chain antibody (scFv), a single-domain antibody, a recombinant heavy-chain-only antibody (VHH), a shark heavy-chain-only antibody (VNAR), a Fv, a Fab, a Fab′, or a F(ab′)₂. In particular embodiments, the antibody, antibody format, or antigen binding portion thereof is a scFv, or variant thereof. In some embodiments, the scFv, or variant thereof, comprises a VL-VH or VH-VL orientation.

In various embodiments, the construct comprises one or more antibody, antibody format, or antigen binding portion thereof directed against an IL-2-responsive cell type marker. In embodiments, the IL-2-responsive cell type is selected from a cytotoxic T lymphocyte (CTL), a natural killer cell (NK), a Thelp cell, and a gamma-delta T cell. In such embodiments, the CTL or NK cell or Thelp cell or gamma-delta T cell marker is selected from one or more of CD3, CD4, CD8, CD28, CD45, CD54, CD56, CD94, CD95, CD226, NKG2D, NKp30, NKp44, NKp46, TCRab, carbonic anhydrase IX (CAIX), 5T4, CD19, CD20, CD22, CD30, CD33, CD38, CD47, CS1, CD138, Lewis-Y, L1-CAM, MUC16, ROR-1, IL13Ralpha2, gp100, prostate stem cell antigen (PSCA), prostate-specific membrane antigen (PSMA), B-cell maturation antigen (BCMA), human papillomavirus type 16 E6 (HPV-16 E6), CD171, folate receptor alpha (FR-alpha), GD2, human epidermal growth factor receptor 2 (HER2), mesothelin, EGFRvlll, fibroblast activation protein (FAP), carcinoembryonic antigen (CEA), vascular endothelial growth factor receptor 2 (VEGF-R2), E-Cadherin, CCR5, CCR6, CD27/TNFRSF7, CD27 Ligand/TNFSF7, CD40/TNFRSF5, CD40 Ligand/TNFSF5, CD83, CD161, CD161/NK1.1, CXC4, Dectin-1/CLEC7A, Fas/TNFRSF6/CD95, Fas Ligand/TNFSF6, Fc gamma RIII (CD16), Fc gamma RIIIA/CD16a, Fc gamma RIIIB/CD16b, ICOS, IL-18 R alpha/IL-1 R5, IL-23R, NKG2D/CD314, NKG2E, Occludin, TCR gamma/delta, TLR2, and TRAIL/TNFSF10.

In embodiments, the method provided by the present disclosure results in endogenous IL-2 being redirected from Treg cells to CTLs and/or NK cells and/or Thelp cells. For example, in embodiments, the method results in neutralization of IL-2 binding and/or interaction with the IL-2 alpha-beta-gamma receptor. In further embodiments, the method reduces IL-2 signaling on a Treg cell, optionally by at least about 10%, 15%, 20%, 25%, 30%, 35%, 40%, 45%, 50%, 55%, 60%, 65%, 70%, 75, 80, 85, 90, or 95%. In various embodiments, the method causes selective proliferation and/or survival of CTLs or NK cells or Thelp cells. In particular embodiments, the method selectively activates CTLs or NK cells or Thelp cells. In various embodiments, the method reduces the ratio of IL-2Ralpha-beta-gamma-mediated signaling to IL-2Rbeta-gamma-mediated signaling, optionally by at least about 10%, 15%, 20%, 25%, 30%, 35%, 40%, 45%, 50%, 55%, 60%, 65%, 70%, 75, 80, 85, 90, or 95%. Further, the method can reduce the ratio of IL-2-modulated Treg cells to IL-2-modulated CTLs or NKs or Thelp cells, optionally by at least about 10%, 15%, 20%, 25%, 30%, 35%, 40%, 45%, 50%, 55%, 60%, 65%, 70%, 75, 80, 85, 90, or 95%.

In some embodiments, the method increases IL-2-mediated STAT signaling on CTLs or NKs or Thelp cells, optionally by at least about 10%, 15%, 20%, 25%, 30%, 35%, 40%, 45%, 50%, 55%, 60%, 65%, 70%, 75, 80, 85, 90, or 95%. In further embodiments, the method decreases IL-2 mediated STAT signaling on Treg cells, optionally by at least about 10%, 15%, 20%, 25%, 30%, 35%, 40%, 45%, 50%, 55%, 60%, 65%, 70%, 75, 80, 85, 90, or 95%. In further embodiments, the method increases IL-2 sensitivity on CTLs or NKs or Thelp cells. In further embodiments, the method decreases IL-2 sensitivity on Treg cells. In additional embodiments, the method causes the ratio of IL-2 sensitivity on Treg cells to CTLs and/or NK cells and/or Thelp cells to be about 1, 2, 3, 4, or 5.

In various embodiments, the method increases anti-tumor activity of IL-2 and decreases activation of Treg cells, optionally by at least about 10%, 15%, 20%, 25%, 30%, 35%, 40%, 45%, 50%, 55%, 60%, 65%, 70%, 75, 80, 85, 90, or 95%. In such embodiments, the decrease of activation of Treg cells results in a decrease of T-cell suppression, optionally by at least about 10%, 15%, 20%, 25%, 30%, 35%, 40%, 45%, 50%, 55%, 60%, 65%, 70%, 75, 80, 85, 90, or 95%.

In embodiments, the construct does not comprise an exogenous wild-type IL-2 molecule.

In some embodiments, the antibody, antibody format, or antigen binding portion thereof directed against an IL-2-responsive cell type marker, optionally a cytotoxic T lymphocyte (CTL) or natural killer cell (NK) or Thelp cell marker or gamma-delta T cell, is an anti-CD8 antibody, antibody format, or antigen binding portion thereof. In such embodiments, the anti-CD8 antibody format is a single-domain antibody, a recombinant heavy-chain-only antibody (VHH), a single-chain antibody (scFv), a shark heavy-chain-only antibody (VNAR), a Fv, a Fab, a Fab′, or a F(ab′)₂. In particular embodiments, the anti-CD8 antibody format is a VHH or scFv. In certain embodiments, the construct comprises a polypeptide having at least about 90%, at least about 93%, at least about 94%, at least about 95%, at least about 96%, at least about 97%, at least about 98%, or at least about 99% amino acid sequence similarity to one of SEQ ID NO: 1-12. In certain embodiments, the construct comprises a polypeptide having at least about 90%, at least about 93%, at least about 94%, at least about 95%, at least about 96%, at least about 97%, at least about 98%, or at least about 99% amino acid sequence similarity to one of SEQ ID NO: 1-12, without a leader sequence and/or His₆ tag. In embodiments, the construct comprises a polypeptide having the amino acid sequence of one of SEQ ID NO: 1-12. In embodiments, the construct comprises a polypeptide having the amino acid sequence of one of SEQ ID NO: 1-12, without a leader sequence and/or His₆ tag. In embodiments, the construct comprises a polypeptide not having a leader sequence. In embodiments, the construct comprises a polypeptide not having a His₆ tag.

In some embodiments, the construct further comprises a Fc domain. In such embodiments, the construct has an increased half-life as compared to a construct without a Fc domain. In embodiments, the Fc domain is a human IgG1 Fc backbone. In some embodiments, the Fc domain comprises effector mutations selected from one or more of L234A, L235A, P331G, and K322Q, or mutations corresponding thereto. In further embodiments, the Fc domain comprises mutations selected from one or more of S354C and T366W, or mutations corresponding thereto. In additional embodiments, the Fc domain further comprises mutations selected from one or more of Y349C, T366S, L368A, and Y407V, or mutations corresponding thereto.

In additional embodiments, the construct further comprises a targeting moiety comprising a recognition domain which specifically binds to a tissue-specific marker. For example, the tissue-specific marker can be selected from endothelial cell surface markers such as ACE, CD14, CD34, CDH5, ENG, ICAM2, MCAM, NOS3, PECAMI, PROCR, SELE, SELP, TEK, THBD, VCAMI, TEM1, TEM8, Clec14A, VWF; smooth muscle cell surface markers such as ACTA2, MYHIO, MYHI 1, MYH9, MYOCD; fibroblast (stromal) cell surface markers such as FAP, ALCAM, CD34, COLIAI, COL1A2, COL3A1, PH-4; epithelial cell surface markers such as CDID, K6IRS2, KRTIO, KRT13, KRT17, KRT18, KRT19, KRT4, KRT5, KRT8, MUCI, TACSTDI; neovasculature markers such as CD13, TFNA, Alpha-v beta-3 (aVB3), E-selectin; and adipocyte surface markers such as ADIPOQ, FABP4, and RETN. In particular embodiments, the tissue is the extracellular matrix (ECM). For example, in embodiments, the ECM marker is selected from a tenascin. In such embodiments, the tenascin is selected from tenascin-C, tenascin-R, tenascin-X, and tenascin-W, a fibronectin, a fibrin, a laminin, and a nidogen/entactin. In particular embodiments, the tenascin is tenascin-CA1.

In various embodiments, the construct is a polypeptide.

In some embodiments, the construct is a nucleic acid. In embodiments, the nucleic acid is or comprises deoxyribonucleic acid (DNA) and/or ribonucleic acid (RNA). In embodiments, the nucleic acid is DNA, optionally selected from linear DNA, DNA fragments, or DNA plasmids. In embodiments, the nucleic acid is an mRNA, optionally comprising one or more non-canonical nucleotides, optionally selected from pseudouridine and 5-methoxyuridine. In particular embodiments, the RNA is modified messenger RNA (mmRNA).

IL-2 Redirection from Activated IL-2-Responsive Cell Types to Treg Cells

In some embodiments, the construct comprises (i) an anti-IL-2 antibody, antibody format, or antigen binding portion thereof, which binds IL-2 in a manner that disrupts or blocks IL-2 interaction with the IL-2 alpha-beta-gamma receptor, and (ii) one or more antibody, antibody format, or antigen binding portion thereof directed against a Treg cell marker.

In embodiments, the Treg cell marker is selected from one or more of CTLA-4, CD25, GITR, CD127, LAG-3, PD-1, and CCR8.

In various embodiments, endogenous IL-2 is redirected from activated CTL or NK or Thelp cells to Treg cells. In embodiments, the method results in neutralization of IL-2 binding and/or interaction with the IL-2 alpha-beta-gamma receptor. In some embodiments, the method reduces IL-2 signaling on an activated CTL or NK or Thelp cell, optionally by at least about 10%, 15%, 20%, 25%, 30%, 35%, 40%, 45%, 50%, 55%, 60%, 65%, 70%, 75, 80, 85, 90, or 95%. In further embodiments, the method causes selective proliferation and/or survival of Treg cells. For example, the method can cause selective activation of Treg cells.

In various embodiments, the method reduces the ratio of IL-2Ralpha-beta-gamma-mediated signaling to IL-2Rbeta-gamma-mediated signaling, optionally by at least about 10%, 15%, 20%, 25%, 30%, 35%, 40%, 45%, 50%, 55%, 60%, 65%, 70%, 75, 80, 85, 90, or 95%. In some embodiments, the method reduces the ratio of IL-2-modulated CTL cells to IL-2-modulated Treg cells, optionally by at least about 10%, 15%, 20%, 25%, 30%, 35%, 40%, 45%, 50%, 55%, 60%, 65%, 70%, 75, 80, 85, 90, or 95%.

In some embodiments, the method increases IL-2-mediated STAT signaling on Treg cells, optionally by at least about 10%, 15%, 20%, 25%, 30%, 35%, 40%, 45%, 50%, 55%, 60%, 65%, 70%, 75, 80, 85, 90, or 95%. In further embodiments, the method decreases IL-2 mediated STAT signaling on CTL or NK or Thelp cells, optionally by at least about 10%, 15%, 20%, 25%, 30%, 35%, 40%, 45%, 50%, 55%, 60%, 65%, 70%, 75, 80, 85, 90, or 95%. In further embodiments, the method increases IL-2 sensitivity on Treg cells, optionally by at least about 10%, 15%, 20%, 25%, 30%, 35%, 40%, 45%, 50%, 55%, 60%, 65%, 70%, 75, 80, 85, 90, or 95%. In further embodiments, the method decreases IL-2 sensitivity on CTLs or NKs or Thelp cells, optionally by at least about 10%, 15%, 20%, 25%, 30%, 35%, 40%, 45%, 50%, 55%, 60%, 65%, 70%, 75, 80, 85, 90, or 95%. In additional embodiments, the method causes the ratio of IL-2 sensitivity on CTLs and/or NK cells and/or Thelp cells to Treg cells to be about 1, 2, 3, 4, or 5.

In some embodiments, the decrease of activation of CTL or NK or Thelp cells results in a dampened immune response, optionally by at least about 10%, 15%, 20%, 25%, 30%, 35%, 40%, 45%, 50%, 55%, 60%, 65%, 70%, 75, 80, 85, 90, or 95%. In embodiments, the construct does not comprise an exogenous wild-type IL-2 molecule.

In some embodiments, the construct further comprises a Fc domain. In such embodiments, the construct has an increased half-life as compared to a construct without a Fc domain. In embodiments, the Fc domain is a human IgG1 Fc backbone. In some embodiments, the Fc domain comprises effector mutations selected from one or more of L234A, L235A, P331G, and K322Q, or mutations corresponding thereto. In further embodiments, the Fc domain comprises mutations selected from one or more of S354C and T366W, or mutations corresponding thereto. In additional embodiments, the Fc domain further comprises mutations selected from one or more of Y349C, T366S, L368A, and Y407V, or mutations corresponding thereto.

In additional embodiments, the construct further comprises a targeting moiety comprising a recognition domain which specifically binds to a tissue-specific marker. For example, the tissue-specific marker can be selected from endothelial cell surface markers such as ACE, CD14, CD34, CDH5, ENG, ICAM2, MCAM, NOS3, PECAMI, PROCR, SELE, SELP, TEK, THBD, VCAMI, TEM1, TEM8, Clec14A, VWF; smooth muscle cell surface markers such as ACTA2, MYHIO, MYHI 1, MYH9, MYOCD; fibroblast (stromal) cell surface markers such as FAP, ALCAM, CD34, COLIAI, COL1A2, COL3A1, PH-4; epithelial cell surface markers such as CDID, K6IRS2, KRTIO, KRT13, KRT17, KRT18, KRT19, KRT4, KRT5, KRT8, MUCI, TACSTDI; neovasculature markers such as CD13, TFNA, Alpha-v beta-3 (αVβ3), E-selectin; and adipocyte surface markers such as ADIPOQ, FABP4, and RETN. In particular embodiments, the tissue is the extracellular matrix (ECM). For example, in embodiments, the ECM marker is selected from a tenascin. In such embodiments, the tenascin is selected from tenascin-C, tenascin-R, tenascin-X, and tenascin-W, a fibronectin, a fibrin, a laminin, and a nidogen/entactin. In particular embodiments, the tenascin is tenascin-CA1.

In various embodiments, the construct is a polypeptide.

In some embodiments, the construct is a nucleic acid. In embodiments, the nucleic acid is or comprises deoxyribonucleic acid (DNA) and/or ribonucleic acid (RNA). In embodiments, the nucleic acid is DNA, optionally selected from linear DNA, DNA fragments, or DNA plasmids. In embodiments, the nucleic acid is an mRNA, optionally comprising one or more non-canonical nucleotides, optionally selected from pseudouridine and 5-methoxyuridine. In particular embodiments, the RNA is modified messenger RNA (mmRNA).

### Tissue-Specific Markers

In various embodiments of the present disclosure, the construct comprises a targeting moiety comprising a recognition domain which specifically binds to a tissue-specific marker.

Non-cellular molecules, which may be secreted by cells and provide structural and biochemical support to the surrounding cells, are increasingly viewed as crucial components of human disease. The tumor microenvironment includes the surrounding blood vessels, signaling molecules, and the extracellular matrix (ECM) or antigens or receptors or non-proteinaceous markers associated therewith. Tumor cells constantly interact with the various components of this microenvironment. For example, tumor cells can release extracellular signals into the microenvironment to promote angiogenesis and peripheral immune tolerance. The extracellular matrix (ECM) has emerged as a critical component of the tumor microenvironment. The ECM is the non-cellular component present within all tissues and organs. Although tightly controlled during embryonic development and organ homeostasis, the ECM becomes dysregulated and disorganized in diseases such as cancer. Abnormal ECM affects cancer progression by directly promoting cellular transformation and metastasis. In addition, ECM anomalies also deregulate the behavior of stromal cells, facilitate tumor-associated angiogenesis and inflammation, and thus lead to generation of a tumorigenic microenvironment.

In some embodiments, the construct comprises a targeting moiety comprising a recognition domain which specifically binds a component of an intact cell or cellular structure. In some embodiments, the target is an extracellular antigen or receptor. In illustrative embodiments, the target of interest is part of a non-cellular structure selected from an extracellular matrix (ECM) antigen or receptor, or a protein or non-proteinaceous marker associated therewith. For example, the targeting moiety may specifically bind to a target selected from a tenascin, a fibronectin, a collagen, a fibrin, a laminin, and a nidogen/entactin. In an exemplary embodiment, the targeting moiety specifically binds to tenascin.

The tissue-specific marker can be selected from endothelial cell surface markers such as ACE, CD14, CD34, CDH5, ENG, ICAM2, MCAM, NOS3, PECAMI, PROCR, SELE, SELP, TEK, THBD, VCAMI, TEM1, TEM8, Clec14A, VWF; smooth muscle cell surface markers such as ACTA2, MYHIO, MYHI 1, MYH9, MYOCD; fibroblast (stromal) cell surface markers such as FAP, ALCAM, CD34, COLIAI, COL1A2, COL3A1, PH-4; epithelial cell surface markers such as CDID, K6IRS2, KRTIO, KRT13, KRT17, KRT18, KRT19, KRT4, KRT5, KRT8, MUCI, TACSTDI; neovasculature markers such as CD13, TFNA, Alpha-v beta-3 (aVB3), E-selectin; and adipocyte surface markers such as ADIPOQ, FABP4, and RETN.

In particular embodiments, the tissue is the extracellular matrix (ECM). For example, in embodiments, the ECM marker is selected from a tenascin. In such embodiments, the tenascin is selected from tenascin-C, tenascin-R, tenascin-X, and tenascin-W, a fibronectin, a fibrin, a laminin, and a nidogen/entactin. In particular embodiments, the tenascin is tenascin-CA1.

### Methods of Treatment

In some embodiments, the present disclosure provides methods for treating cancer in a subject, the methods comprising administering to the subject a construct comprising (i) an anti-IL-2 antibody, antibody format, or antigen binding portion thereof, which binds IL-2 in a manner that disrupts or blocks IL-2 interaction with the IL-2 alpha-beta-gamma receptor, and (ii) one or more antibody, antibody format, or antigen binding portion thereof directed against an IL-2-responsive cell type marker, wherein the anti-IL-2 antibody, antibody format, or antigen binding portion thereof and the one or more antibody, antibody format, or antigen binding portion thereof directed against an IL-2-responsive cell type marker are linked. In embodiments, the methods for treating cancer comprise reducing, mitigating, or eliminating endogenous IL-2 signaling and/or binding to the IL-2 alpha-beta-gamma receptor in the subject. In embodiments, the methods for treating cancer comprise redirecting endogenous IL-2 signaling from the IL-2 alpha-beta-gamma receptor to the IL-2 beta-gamma receptor on an antigen-expressing cell in the subject.

In embodiments, the cancer is selected form one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses; edema (e.g. that associated with brain tumors); and Meigs' syndrome.

In some embodiments, the present disclosure provides methods for treating an auto-immune disease or disorder or an inflammation-related disorder in a subject, the methods comprising administering to the subject a construct comprising (i) an anti-IL-2 antibody, antibody format, or antigen binding portion thereof, which binds IL-2 in a manner that disrupts or blocks IL-2 interaction with the IL-2 alpha-beta-gamma receptor, and (ii) one or more antibody, antibody format, or antigen binding portion thereof directed against a Treg cell marker, wherein the anti-IL-2 antibody, antibody format, or antigen binding portion thereof and the one or more antibody, antibody format, or antigen binding portion thereof directed against a Treg cell marker are linked. In embodiments, the methods for treating an auto-immune disease or disorder or an inflammation-related disorder comprise reducing, mitigating, or eliminating endogenous IL-2 signaling and/or binding to the IL-2 alpha-beta-gamma receptor in the subject. In embodiments, the methods for treating an auto-immune disease or disorder or an inflammation-related disorder comprise redirecting endogenous IL-2 signaling from the IL-2 alpha-beta-gamma receptor to the IL-2 beta-gamma receptor on an antigen-expressing cell in the subject.

In embodiments, the auto-immune disease or disorder is selected form one or more of multiple sclerosis, celiac disease, rheumatoid arthritis, systemic lupus erythematosus, Sjogren's syndrome, polymyalgia rheumatica, ankylosing spondylitis, type 1 diabetes, vasculitis, temporal arteritis, Graves disease, dermatomyositis, Addison disease, Hashimoto thyroiditis, Myasthenia gravis, and pernicious anemia.

In embodiments, the inflammation-related disorder is selected from one or more of inflammation, acute inflammation, chronic inflammation, respiratory disease, atherosclerosis, restenosis, asthma, allergic rhinitis, atopic dermatitis, septic shock, rheumatoid arthritis, inflammatory bowel disease, inflammatory pelvic disease, pain, ocular inflammatory disease, celiac disease, Leigh Syndrome, Glycerol Kinase Deficiency, Familial eosinophilia (FE), autosomal recessive spastic ataxia, laryngeal inflammatory disease; Tuberculosis, Chronic cholecystitis, Bronchiectasis, Silicosis and other pneumoconiosis.

### Production of Anti-IL-2 Constructs

Methods for producing the constructs of the disclosure (e.g., the anti-IL-2 antibody, antibody format, or antigen binding portion thereof and the one or more antibody, antibody format, or antigen binding portion thereof directed against an IL-2-responsive cell type marker and the linker) are described herein. For example, DNA sequences encoding the constructs of the disclosure (e.g., DNA sequences encoding the anti-IL-2 antibody, antibody format, or antigen binding portion thereof and the one or more antibody, antibody format, or antigen binding portion thereof directed against an IL-2-responsive cell type marker and the linker) can be chemically synthesized using methods known in the art. Synthetic DNA sequences can be ligated to other appropriate nucleotide sequences, including, e.g., expression control sequences, to produce gene expression constructs encoding the desired anti-IL-2 constructs. Accordingly, in various embodiments, the present disclosure provides for isolated nucleic acids comprising a nucleotide sequence encoding the constructs of the present disclosure.

Nucleic acids encoding the constructs of the disclosure can be incorporated (ligated) into expression vectors, which can be introduced into host cells through transfection, transformation, or transduction techniques. For example, nucleic acids encoding the constructs of the disclosure can be introduced into host cells by retroviral transduction. Illustrative host cells are E. coli cells, Chinese hamster ovary (CHO) cells, human embryonic kidney 293 (HEK 293) cells, Hela cells, baby hamster kidney (BHK) cells, monkey kidney cells (COS), human hepatocellular carcinoma cells (e.g., Hep G2), insect Sf9 cells, and myeloma cells. Transformed host cells can be grown under conditions that permit the host cells to express the genes that encode the constructs of the disclosure. Accordingly, in various embodiments, the present disclosure provides expression vectors comprising nucleic acids that encode the constructs of the disclosure. In various embodiments, the present disclosure additionally provides host cells comprising such expression vectors.

### Subjects and Indications

In some embodiments, the subject is afflicted with cancer. In embodiments, the cancer is a solid tumor or a blood cancer. In some embodiments, the cancer is selected form one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses; edema (e.g. that associated with brain tumors); and Meigs' syndrome.

In some embodiments, the subject is afflicted with an auto-immune disease or disorder. In embodiments, the auto-immune disease or disorder is selected form one or more of multiple sclerosis, celiac disease, rheumatoid arthritis, systemic lupus erythematosus, Sjogren's syndrome, polymyalgia rheumatica, ankylosing spondylitis, type 1 diabetes, vasculitis, temporal arteritis, Graves disease, dermatomyositis, Addison disease, Hashimoto thyroiditis, Myasthenia gravis, and pernicious anemia.

In some embodiments, the subject is afflicted with an inflammation-related disorder. In embodiments, the inflammation-related disorder is selected from one or more of inflammation, acute inflammation, chronic inflammation, respiratory disease, atherosclerosis, restenosis, asthma, allergic rhinitis, atopic dermatitis, septic shock, rheumatoid arthritis, inflammatory bowel disease, inflammatory pelvic disease, pain, ocular inflammatory disease, celiac disease, Leigh Syndrome, Glycerol Kinase Deficiency, Familial eosinophilia (FE), autosomal recessive spastic ataxia, laryngeal inflammatory disease; Tuberculosis, Chronic cholecystitis, Bronchiectasis, Silicosis and other pneumoconiosis

### Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

As used herein, the term “effective amount” refers to a quantity sufficient to achieve a desired therapeutic and/or prophylactic effect, e.g., an amount which results in the prevention of, or a decrease in a disease or disorder or one or more signs or symptoms associated with a disease or disorder. In the context of therapeutic or prophylactic applications, the amount of a composition administered to the subject will depend on the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compounds may be administered to a subject having one or more signs or symptoms of a disease or disorder. As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim embodiments of the disclosure, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology. The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

### EXAMPLES

### Example 1: Construction, Production and Purification of Anti-IL-2 Constructs

In a series of fusion proteins, scFv variants (in the VH-VL or VL-VH orientation) of the neutralizing antibodies NARA1 and TCB2 were coupled to a scFv variant of the CD8 alpha-specific OKT8 antibody (in the VH-VL orientation).

NARA1 is a monoclonal antibody to human IL-2 that acts like a high-affinity CD25 mimic, thus minimizing association of IL-2 with CD25. See Arenas-Ramirez et al., “Improved cancer immunotherapy by a CD25-mimobody conferring selectivity to human interleukin-2,” Sci Trans/Med 8 (367), 2016, the entire contents of which are incorporated by reference. TCB2 is an anti-human IL-2 (hIL-2) monoclonal antibody that selectively stimulates CD8 T and NK cells without overtly activating Tregs. See Lee et al., “TCB2, a new anti-human interleukin-2 antibody, facilitates heterodimeric IL-2 receptor signaling and improves anti-tumor immunity,” Oncoimmunology 9 (1): 1681869, 2019, the entire contents of which are incorporated by reference.

The following constructs were used:
1. NARA1 scFv_v1-OKT8 scFv: anti-IL-2 Ab NARA1 scFv_VH-VL-3*GGGGS-anti-CD8 OKT8 scFv_VH-VL-6*His
2. NARA1 scFv_v2-OKT8 scFv: anti-IL-2 Ab NARA1 scFv_VL-VH-3*GGGGS-anti-CD8 OKT8 scFv_VH-VL-6*His
3. TCB2 scFv_v1-OKT8 scFv: anti-IL-2 Ab TCB2 scFv_VH-VL-3*GGGGS-anti-CD8 OKT8 scFv_VH-VL-6*His
4. TCB2 scFv_v2-OKT8 scFv: anti-IL-2 Ab TCB2 scFv_VL-VH-3*GGGGS-anti-CD8 OKT8 scFv_VH-VL-6*His
5. NARA1 scFv_v1: anti-IL-2 Ab NARA1 scFv_VH-VL-6*His
6. NARA1 scFv_v2: anti-IL-2 Ab NARA1 scFv_VL-VH-6*His
7. TCB2 scFv_v1: anti-IL-2 Ab TCB2 scFv_VH-VL-6*His
8. TCB2 scFv_v2: anti-IL-2 Ab TCB2 scFv_VL-VH-6*His
9 OKT8 scFv: anti-CD8 OKT8 scFv_VH-VL-6*His

Different anti-IL-2 construct variants were transfected in ExpiCHO cells (ThermoFisher) according to the manufacturer's instructions. One week after transfection, supernatant was collected, and cells removed by centrifugation. Recombinant proteins were purified based on the His-tag (HisTrap Excel column; Cytiva) and by subsequent size exclusion chromatography (Superdex 200 increase HiScale 16/40 column, Cytiva), both on an Akta purifier (GE Healthcare). Concentrations were measured with a spectrophotometer (NanoDrop instrument, Thermo Scientific) and purity estimated on SDS-PAGE.

### Example 2: Anti-IL-2 Constructs Bind Both IL-2 and CD8 Alpha

Analysis by bio-layer interferometry (BLI) on an Octet RED96 instrument (ForteBio) shows that the bi-specific anti-IL-2 constructs bind to IL-2 on the one hand, and to CD8 alpha on the other.

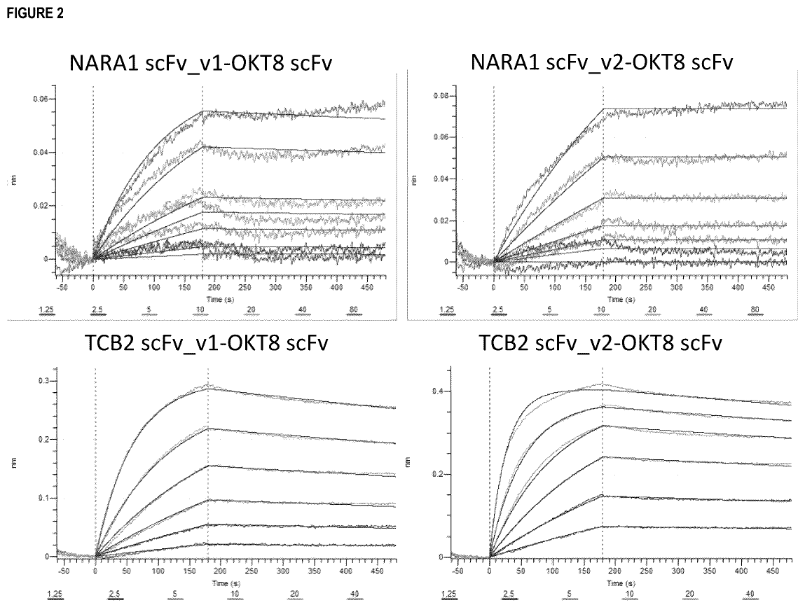

In brief, recombinant IL-2 (Acro Biosystems) was biotinylated using the Pierce Antibody Biotinylation Kit for IP (ThermoFisher) and loaded on Streptavidin sensors. Association and dissociation of seven concentrations of each of the bi-specific anti-IL-2 constructs were monitored and used to calculate the association and dissociation constants and hence affinity using the Octet software. Sensorgrams and calculated binding kinetics provided in FIG. 2 illustrate that (i) all anti-IL-2 constructs clearly bind to biotinylated IL-2; (ii) for both anti-IL-2 antibody scFv variants, the VL-VH configuration (referred to as scFv_v2) seems to work best; and (iii) TCB2-based anti-IL-2 constructs tend to associate faster, while NARA1 anti-IL-2 constructs dissociate slower.

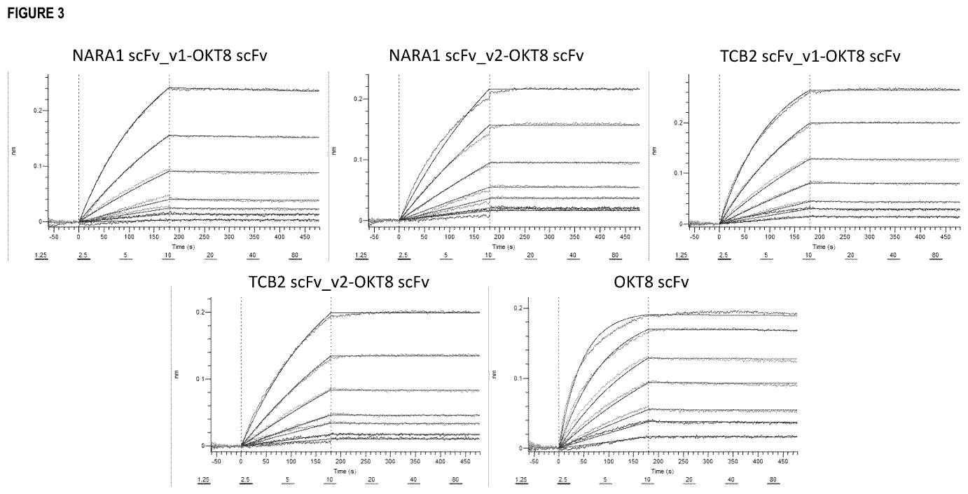

Binding to OKT8 was tested in a similar manner: biotinylated CD8 was loaded onto a Streptavidin sensor. Bi-specific anti-IL-2 constructs or the isolated OKT8 scFv were used as analyte. Sensorgrams and binding characteristics are shown in FIG. 3 and illustrate that binding for all molecules is very similar.

### Example 3: Anti-IL-2 Constructs Interfere with Binding of IL-2 to the IL-2Ralpha Chain

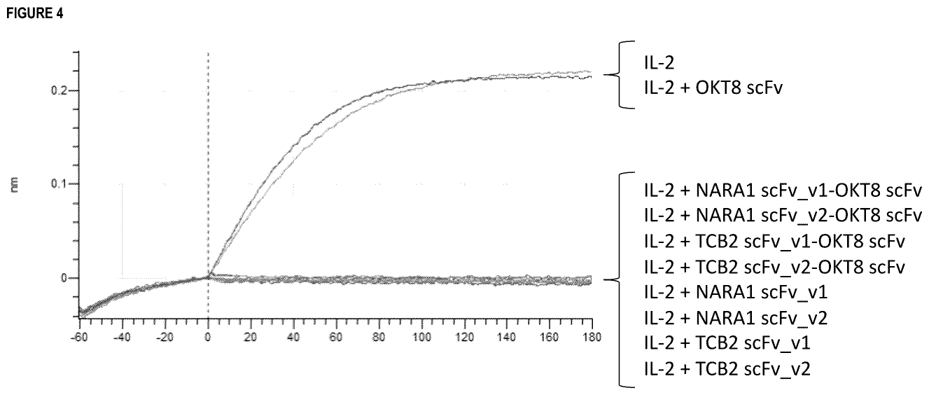

The ability of the anti-IL-2 constructs to interfere with binding of IL-2 to the IL-2R alpha was evaluated in a BLI experimental set-up. Recombinant CD25 (IL-2R alpha) was biotinylated using the Pierce Antibody Biotinylation Kit for IP (ThermoFisher) and immobilized on Streptavidin sensors. IL-2 (25 nM) or IL-2 (25 nM) 30 min pre-incubated with the different anti-IL-2 constructs (250 nM) was allowed to bind to IL-2Ralpha loaded sensors. Data in FIG. 4 show clear binding of IL-2 on its own, and that all tested anti-IL-2 constructs or the anti-IL-2 antibody scFv's clearly blocked this interaction. OKT8, as expected, does not influence this interaction and results in an association comparable to IL-2 alone.

### Example 4: Anti-IL-2 Constructs Re-route IL-2 Activity from Treg to CD8 Positive Cells

Anti-IL-2 constructs were tested for their effects on STAT5 phosphorylation in CD8 positive T cells (CD8⁺), as compared to Tregs defined as CD4⁺CD25⁺FoxP3⁺.

In brief, PBMCs from buffy coats of healthy donors were isolated using density gradient centrifugation using Lymphoprep (StemCell technologies). A serial dilution of wild type recombinant IL-2 was pre-incubated (1 hour at 37° C.) with a fixed concentration (10 μg/ml) of (i) NARA1 scFv_v2-OKT8 scFv; (ii) TCB2 scFv_v2-OKT8 scFv, (iii) NARA1 scFv_v2; (iv) TCB2 scFv_v2; (v) OKT8 scFv, or (vi) without competitor. These mixes were used to stimulate the isolated PBMCs for 30 minutes at 37 degrees Celsius. After centrifugation, cells were resuspended in Lyse/Fix buffer (BD Biosciences) and further incubated for 10 minutes at 37 degrees Celsius. Cells were washed and incubated with human FcR Blocking Reagent (Miltenyi Biotec) and stained with anti-CD25 and anti-CD8 for 30 minutes at room temperature. Cells were subsequently permeabilized using the Perm Buffer III (BD Biosciences) at 4 degrees Celsius for 30 minutes. Cells were finally stained with anti-CD3, anti-CD4, anti-FoxP3, and anti-pSTAT5 for 1 hour. Samples were acquired on a MACSQuant X instrument (Miltenyi Biotec) and analyzed using the FlowLogic software (Miltenyi Biotec).

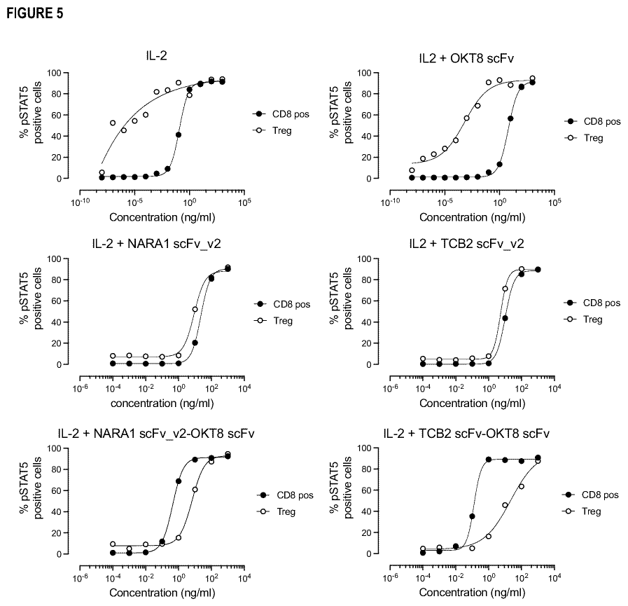

The data presented in FIG. 5 clearly illustrate that:
i. Treg cells are much more sensitive to IL-2 compared to CD8⁺ cells (upper-left panel);
ii. The anti-CD8 alpha OKT8 scFv does not affect IL-2 signaling in Tregs, or in CD8⁺ cells (upper-right panel);
iii. The anti-IL-2 NARA1 and TCB2 scFv's (which block binding of IL-2 to the IL-2Ralpha) reduce IL-2 signaling in Treg and CD8⁺ to the beta: gamma common IL-2R complex. This results in a comparable sensitivity to IL-2 for both cell-types (middle panels);
iv. CD8 targeting of the NARA1 and TCB2 scFv's increase signaling in CD8⁺ cells, but not in Treg's (lower panels).

Together these data show that neutralization of the IL-2: IL-2Ralpha interaction reduces IL-2 signaling to a similar level in CD8⁺ and Treg cells. Additional CD8alpha targeting results in an increased STAT5 phosphorylation in CD8alpha expressing cells. The data suggest that the present disclosure allows for re-directing IL-2 signaling from the highly sensitive Treg cells to CD8+ cells with intermediate to low affinity for IL-2 and hence strongly revert the IL-2 preference for Treg signaling to a preference for CD8+ cell signaling.

### Example 5: Anti-IL-2 Constructs in an Fc-context Re-direct IL-2 Signaling

The anti-IL-2 construct used in the previous example is the result of genetically fusing an anti-IL-2 scFv (NARA1 or TCB2) and an anti-CD8alpha scFv (OKT8). To increase the half-life of these molecules, the possibility of adding the scFv's to an Fc backbone was explored.

The scFv's were cloned (in the pcDNA3.4 vector) via a flexible 20*GGS-linker to a heterodimeric, ‘knob-in-hole’ human IgG1 Fc backbone. Fc sequences contain the L234A_L235A_P331G effector mutations and the ‘hole’ modifications Y349C_T366S_L368A_Y407V (in sequence hFc3) or ‘knob’ mutations S354C_T366W (in sequence hFc4). The TCB2 scFv_v2 was cloned N-terminally of the hole chain (see sequence TCB2 scFv_v2-hFc3 below), and OKT8 scFv was cloned C-terminally on the knob Fc-chain (sequence hFc4-OKT8 scFv). To produce this ‘knob-in-hole’ Fc-Anti-IL-2 construct, a combination of both ‘hole’ and ‘knob’ plasmids was transfected in ExpiCHO cells (ThermoFisher) according to the manufacturer's instructions. Seven days post transfection, recombinant proteins were purified based on protein A binding properties (Hitrap MabSelect SuRe column, GE Healthcare) and by subsequent size exclusion chromatography (Superdex 200 increase HiScale 16/40 column, GE Healthcare), both on an Akta purifier (GE Healthcare). Concentrations were measured with a spectrophotometer (NanoDrop instrument, Thermo Scientific), and purity was estimated on SDS-PAGE.

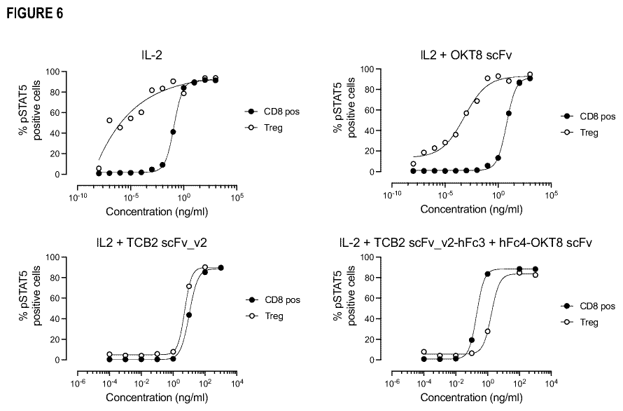

The resulting Fc anti-IL-2 construct or the scFv controls were tested for effect on IL-2 driven STAT5 phosphorylation in CD8⁺ and Treg cells (FIG. 6). As expected, the OKT8 scFv does not affect IL-2 signaling in either cell type (upper-right panel), while IL-2 sensitivity becomes essentially the same when pre-treated with TCB2 scFv (lower-left panel). On the other hand, in the presence of the Fc Anti-IL-2 construct IL-2, STAT5 phosphorylation increased by 10-fold in CD8⁺ over Treg cells (lower-right panel), thus showing a reversion of the higher sensitivity of Treg for IL-2 to a higher sensitivity of CD8+ cells.

### Example 6: CD8 Alpha VHH Targeting of Anti-IL-2 Constructs

In this example, the potential of a CD8 VHH targeted anti-IL-2 construct was evaluated.

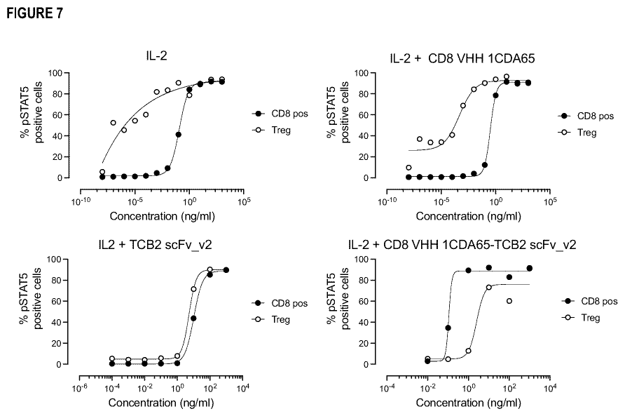

Sequences encoding the CD8 VHH 1CDA65 and the TCB2 scFv_v2 were fused via a flexible 3*GGGGS linker in the pcDNA3.4 vector (resulting sequence CD8 VHH 1CDA65-TCB2 scFv_v2-6*His). Production, purification, and quantification of STAT5 phosphorylation in CD8⁺ compared to Treg cells were as described above. The data in FIG. 7 clearly illustrate that (i) the CD8 VHH does not interfere with IL-2 signaling (upper-right panel); and that (ii) a CD8 VHH based anti-IL-2 construct is able re-direct IL-2 activity from Treg to CD8+ cells (lower-right panel).

The following amino acid sequences were used in the previous Working Examples. Underlined text depicts leader sequences; bolded text depicts linker sequences; and underlined and bolded text depicts His₆ tag.

1.
anti-IL-2 Ab NARA1 scFv_VH-VL-3*GGGGS-anti-CD8 OKT8 scFv_VH-VL-6*His
(NARA1 scFv_v1-OKT8 scFv)
(SEQ ID NO: 1)
MEFGLSWLFLVAILKGVQCQVQLQQSGAELVRPGTSVKVSCKASGYAFTNYLIEWVKQRPGQGLEWIG
VINPGSGGTNYNEKFKGKATLTADKSSSTAYMQLSSLTSDDSAVYFCARWRGDGYYAYFDVWGAGTTV
TVSSGGGGSGGGGSGGGGSDIVLTQSPASLAVSLGQRATISCKASQSVDYDGDSYMNWYQQKPGQPPK
LLIYAASNLESGIPARFSGSGSGTDFTLNIHPVEEEDAATYYCQQSNEDPYTFGGGTKLEIKGGGGSG
GGGSGGGGSEVQLVQSGAEVKKPGASVKVSCKASGFNIKDTYIHWVRQAPGQGLEWIGRIDPANDNTL
YASKFQGRATITADTSTSTAYLELSSLRSEDTAVYYCGRGYGYYVFDHWGQGTLVTVSSGGGGSGGGG
SGGGGSDVQITQSPSSLSASVGDRVTITCRTSRSISQYLAWYQEKPGKTNKLLIYSGSTLQSGIPSRF
SGSGSGTDFTLTISSLQPEDFATYYCQQVNEFPPTFGQGTKVEIKHHHHHH*
2.
anti-IL-2 Ab NARA1 scFv_VL-VH-3*GGGGS-anti-CD8 OKT8 scFv_VH-VL-6*His
(NARA1 scFv_v2-OKT8 scFv)
(SEQ ID NO: 2)
MEFGLSWLFLVAILKGVQCDIVLTQSPASLAVSLGQRATISCKASQSVDYDGDSYMNWYQQKPGQPPK
LLIYAASNLESGIPARFSGSGSGTDFTLNIHPVEEEDAATYYCQQSNEDPYTFGGGTKLEIKGGGGSG
GGGSGGGGSQVQLQQSGAELVRPGTSVKVSCKASGYAFTNYLIEWVKQRPGQGLEWIGVINPGSGGTN
YNEKFKGKATLTADKSSSTAYMQLSSLTSDDSAVYFCARWRGDGYYAYFDVWGAGTTVTVSSGGGGSG
GGGSGGGGSEVQLVQSGAEVKKPGASVKVSCKASGFNIKDTYIHWVRQAPGQGLEWIGRIDPANDNTL
YASKFQGRATITADTSTSTAYLELSSLRSEDTAVYYCGRGYGYYVFDHWGQGTLVTVSSGGGGSGGGG
SGGGGSDVQITQSPSSLSASVGDRVTITCRTSRSISQYLAWYQEKPGKTNKLLIYSGSTLQSGIPSRF
SGSGSGTDFTLTISSLQPEDFATYYCQQVNEFPPTFGQGTKVEIKHHHHHH*
3.
anti-IL-2 Ab TCB2 scFv_VH-VL-3*GGGGS-anti-CD8 OKT8 scFv_VH-VL-6*His
(TCB2 scFv_v1-OKT8 scFv)
(SEQ ID NO: 3)
MEFGLSWLFLVAILKGVQCEVQLQQSGAELARPGASVKLSCKASGYTFTTYWIQWVKQRPGQGLEWIG
AIYPGDGDTRYIQNFKGKATLTADKSSSTAYMQLSSLASEDSAVYYCARSLATRGFYAMDYWGQGTSV
TVSSGGGGSGGGGSGGGGSDIVMTQSPASLSMAIGEKVTIRCITSTDIDDDMNWYQQKPGEPPKLLIS
EGNTLRPGVPSRFSSSGYGTDFVFTIENMLSEDVADYYCLQSDNLPYTFGGGTKLEIKGGGGSGGGGS
GGGGSEVQLVQSGAEVKKPGASVKVSCKASGFNIKDTYIHWVRQAPGQGLEWIGRIDPANDNTLYASK
FQGRATITADTSTSTAYLELSSLRSEDTAVYYCGRGYGYYVFDHWGQGTLVTVSSGGGGSGGGGSGGG
GSDVQITQSPSSLSASVGDRVTITCRTSRSISQYLAWYQEKPGKTNKLLIYSGSTLQSGIPSRFSGSG
SGTDFTLTISSLQPEDFATYYCQQVNEFPPTFGQGTKVEIKHHHHHH*
4.
anti-IL-2 Ab TCB2 scFv_VL-VH-3*GGGGS-anti-CD8 OKT8 scFv_VH-VL-6*His
(TCB2 scFv_v2-OKT8 scFv)
(SEQ ID NO: 4)
MEFGLSWLFLVAILKGVQCDIVMTQSPASLSMAIGEKVTIRCITSTDIDDDMNWYQQKPGEPPKLLIS
EGNTLRPGVPSRFSSSGYGTDFVFTIENMLSEDVADYYCLQSDNLPYTFGGGTKLEIKGGGGSGGGGS
GGGGSEVQLQQSGAELARPGASVKLSCKASGYTFTTYWIQWVKQRPGQGLEWIGAIYPGDGDTRYIQN
FKGKATLTADKSSSTAYMQLSSLASEDSAVYYCARSLATRGFYAMDYWGQGTSVTVSSGGGGSGGGGS
GGGGSEVQLVQSGAEVKKPGASVKVSCKASGENIKDTYIHWVRQAPGQGLEWIGRIDPANDNTLYASK
FQGRATITADTSTSTAYLELSSLRSEDTAVYYCGRGYGYYVFDHWGQGTLVTVSSGGGGSGGGGSGGG
GSDVQITQSPSSLSASVGDRVTITCRTSRSISQYLAWYQEKPGKTNKLLIYSGSTLQSGIPSRFSGSG
SGTDFTLTISSLQPEDFATYYCQQVNEFPPTFGQGTKVEIKHHHHHH*
5.
anti-IL-2 Ab NARA1 scFv_VH-VL-6*His (NARA1 scFv_v1)
(SEQ ID NO: 5)
MEFGLSWLFLVAILKGVQCQVQLQQSGAELVRPGTSVKVSCKASGYAFTNYLIEWVKQRPGQGLEWIG
VINPGSGGTNYNEKFKGKATLTADKSSSTAYMQLSSLTSDDSAVYFCARWRGDGYYAYFDVWGAGTTV
TVSSGGGGSGGGGSGGGGSDIVLTQSPASLAVSLGQRATISCKASQSVDYDGDSYMNWYQQKPGQPPK
LLIYAASNLESGIPARFSGSGSGTDFTLNIHPVEEEDAATYYCQQSNEDPYTFGGGTKLEIKHHHHHH
*
6.
anti-IL-2 Ab NARA1 scFv_VL-VH-6*His (NARA1 scFv_v2)
(SEQ ID NO: 6)
MEFGLSWLFLVAILKGVQCDIVLTQSPASLAVSLGQRATISCKASQSVDYDGDSYMNWYQQKPGQPPK
LLIYAASNLESGIPARFSGSGSGTDFTLNIHPVEEEDAATYYCQQSNEDPYTFGGGTKLEIKGGGGSG
GGGSGGGGSQVQLQQSGAELVRPGTSVKVSCKASGYAFTNYLIEWVKQRPGQGLEWIGVINPGSGGTN
YNEKFKGKATLTADKSSSTAYMQLSSLTSDDSAVYFCARWRGDGYYAYFDVWGAGTTVTVSSHHHHHH
*
7.
anti-IL-2 Ab TCB2 scFv_VH-VL-6*His (TCB2 scFv_v1)
(SEQ ID NO: 7)
MEFGLSWLFLVAILKGVQCEVQLQQSGAELARPGASVKLSCKASGYTFTTYWIQWVKQRPGQGLEWIG
AIYPGDGDTRYIQNFKGKATLTADKSSSTAYMQLSSLASEDSAVYYCARSLATRGFYAMDYWGQGTSV
TVSSGGGGSGGGGSGGGGSDIVMTQSPASLSMAIGEKVTIRCITSTDIDDDMNWYQQKPGEPPKLLIS
EGNTLRPGVPSRFSSSGYGTDFVFTIENMLSEDVADYYCLQSDNLPYTFGGGTKLEIKHHHHHH*
8.
anti-IL-2 Ab TCB2 scFv_VL-VH-6*His (TCB2 scFv_v2)
(SEQ ID NO: 8)
MEFGLSWLFLVAILKGVQCDIVMTQSPASLSMAIGEKVTIRCITSTDIDDDMNWYQQKPGEPPKLLIS
EGNTLRPGVPSRFSSSGYGTDFVFTIENMLSEDVADYYCLQSDNLPYTFGGGTKLEIKGGGGSGGGGS
GGGGSEVQLQQSGAELARPGASVKLSCKASGYTFTTYWIQWVKQRPGQGLEWIGAIYPGDGDTRYIQN
FKGKATLTADKSSSTAYMQLSSLASEDSAVYYCARSLATRGFYAMDYWGQGTSVTVSSHHHHHH*
9.
anti-CD8 OKT8 scFv_VH-VL-6*His (OKT8 scFv)
(SEQ ID NO: 9)
MEFGLSWLFLVAILKGVQCEVQLVQSGAEVKKPGASVKVSCKASGFNIKDTYIHWVRQAPGQGLEWIG
RIDPANDNTLYASKFQGRATITADTSTSTAYLELSSLRSEDTAVYYCGRGYGYYVFDHWGQGTLVTVS
SGGGGSGGGGSGGGGSDVQITQSPSSLSASVGDRVTITCRTSRSISQYLAWYQEKPGKTNKLLIYSGS
TLQSGIPSRFSGSGSGTDFTLTISSLQPEDFATYYCQQVNEFPPTFGQGTKVEIKHHHHHH*
10.
TCB2 scFv_v2-hFc3
(SEQ ID NO: 10)
MEFGLSWLFLVAILKGVQCDIVMTQSPASLSMAIGEKVTIRCITSTDIDDDMNWYQQKPGEPPKLLIS
EGNTLRPGVPSRFSSSGYGTDFVFTIENMLSEDVADYYCLQSDNLPYTFGGGTKLEIKGGGGSGGGGS
GGGGSEVQLQQSGAELARPGASVKLSCKASGYTFTTYWIQWVKQRPGQGLEWIGAIYPGDGDTRYIQN
FKGKATLTADKSSSTAYMQLSSLASEDSAVYYCARSLATRGFYAMDYWGQGTSVTVSSGGSGGSGGSG
GSGGSDKTHTCPPCPAPEAAGGPSVFLFPPKPKDTLMISRTPEVTCVVVDVSHEDPEVKENWYVDGVE
VHNAKTKPREEQYNSTYRVVSVLTVLHQDWLNGKEYKCKVSNKALPAGIEKTISKAKGQPREPQVCTL
PPSRDELTKNQVSLSCAVKGFYPSDIAVEWESNGQPENNYKTTPPVLDSDGSFFLVSKLTVDKSRWQQ
GNVFSCSVMHEALHNHYTQKSLSLSPGK*
11.
hFc4-OKT8 scFv
(SEQ ID NO: 11)
MDMRVPAQLLGLLLLWLRGARCDKTHTCPPCPAPEAAGGPSVFLFPPKPKDTLMISRTPEVTCVVVDV
SHEDPEVKFNWYVDGVEVHNAKTKPREEQYNSTYRVVSVLTVLHQDWLNGKEYKCKVSNKALPAGIEK
TISKAKGQPREPQVYTLPPCRDELTKNQVSLWCLVKGFYPSDIAVEWESNGQPENNYKTTPPVLDSDG
SFFLYSKLTVDKSRWQQGNVFSCSVMHEALHNHYTQKSLSLSPGKGGSGGSGGSGGSGGSGGSGGSGG
SGGSGGSEVQLVQSGAEVKKPGASVKVSCKASGENIKDTYIHWVRQAPGQGLEWIGRIDPANDNTLYA
SKFQGRATITADTSTSTAYLELSSLRSEDTAVYYCGRGYGYYVFDHWGQGTLVTVSSGGGGSGGGGSG
GGGSDVQITQSPSSLSASVGDRVTITCRTSRSISQYLAWYQEKPGKTNKLLIYSGSTLQSGIPSRFSG
SGSGTDFTLTISSLQPEDFATYYCQQVNEFPPTFGQGTKVEIK*
12.
CD8 VHH 1CDA65-TCB2 scFv_v2-6*His
(SEQ ID NO: 12)
MEFGLSWLFLVAILKGVQCDVQLQESGGGLVQPGGSLRLSCAASGSIFSINVMGWYRQTPGKERELVA
KITNFGITSYADSAQGRFTISRGNAKNTVYLQMNSLKPEDTAVYYCNLDTTGWGPPPYQYWGQGTQVT
VSSGGGGSGGGGSGGGGSDIVMTQSPASLSMAIGEKVTIRCITSTDIDDDMNWYQQKPGEPPKLLISE
GNTLRPGVPSRFSSSGYGTDFVFTIENMLSEDVADYYCLQSDNLPYTFGGGTKLEIKGGGGSGGGGSG
GGGSEVQLQQSGAELARPGASVKLSCKASGYTFTTYWIQWVKQRPGQGLEWIGAIYPGDGDTRYIQNF
KGKATLTADKSSSTAYMQLSSLASEDSAVYYCARSLATRGFYAMDYWGQGTSVTVSSHHHHHH*

### EQUIVALENTS

While the disclosure has been described in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior disclosure.

As used herein, all headings are simply for organization and are not intended to limit the disclosure in any manner. The content of any individual section may be equally applicable to all sections.

What is claimed is:

1. A method of reducing, mitigating, or eliminating endogenous IL-2 signaling and/or binding to the IL-2 alpha-beta-gamma receptor in a subject, the method comprising administering to the subject a construct comprising
(i) an anti-IL-2 antibody, antibody format, or antigen binding portion thereof, which binds IL-2 in a manner that disrupts or blocks IL-2 interaction with the IL-2 alpha-beta-gamma receptor, and
(ii) one or more antibody, antibody format, or antigen binding portion thereof directed against an IL-2-responsive cell type marker,
wherein the anti-IL-2 antibody, antibody format, or antigen binding portion thereof and the one or more antibody, antibody format, or antigen binding portion thereof directed against an IL-2-responsive cell type marker are linked.

2. A method of redirecting endogenous IL-2 signaling from the IL-2 alpha-beta-gamma receptor to the IL-2 beta-gamma receptor on an antigen-expressing cell in a subject, the method comprising administering to the subject a construct comprising:
(i) an anti-IL-2 antibody, antibody format, or antigen binding portion thereof, which binds IL-2 in a manner that disrupts or blocks IL-2 interaction with the IL-2 alpha-beta-gamma receptor, and
(ii) one or more antibody, antibody format, or antigen binding portion thereof directed against an IL-2-responsive cell type marker,
wherein the anti-IL-2 antibody, antibody format, or antigen binding portion thereof and the one or more antibody, antibody format, or antigen binding portion thereof directed against an IL-2-responsive cell type marker are linked.

3. The method of claim 1 or 2, wherein the IL-2-responsive cell type is selected from a cytotoxic T lymphocyte (CTL), a natural killer cell (NK), a Thelp cell, and a gamma-delta T cell.

4. The method of any one of claims 1-3, wherein the endogenous IL-2 is redirected from Treg cells to CTLs and/or NK cells and/or Thelp cells and/or gamma-delta T cells.

5. The method of any one of claims 1-4, wherein the CTL or NK cell or Thelp cell or gamma-delta T cell marker is selected from one or more of CD3, CD4, CD8, CD28, CD45, CD54, CD56, CD94, CD95, CD226, NKG2D, NKp30, NKp44, NKp46, TCRab, carbonic anhydrase IX (CAIX), 5T4, CD19, CD20, CD22, CD30, CD33, CD38, CD47, CS1, CD138, Lewis-Y, L1-CAM, MUC16, ROR-1, IL13Ralpha2, gp100, prostate stem cell antigen (PSCA), prostate-specific membrane antigen (PSMA), B-cell maturation antigen (BCMA), human papillomavirus type 16 E6 (HPV-16 E6), CD171, folate receptor alpha (FR-alpha), GD2, human epidermal growth factor receptor 2 (HER2), mesothelin, EGFRvIII, fibroblast activation protein (FAP), carcinoembryonic antigen (CEA), vascular endothelial growth factor receptor 2 (VEGF-R2), E-Cadherin, CCR5, CCR6, CD27/TNFRSF7, CD27 Ligand/TNFSF7, CD40/TNFRSF5, CD40 Ligand/TNFSF5, CD83, CD161, CD161/NK1.1, CXC4, Dectin-1/CLEC7A, Fas/TNFRSF6/CD95, Fas Ligand/TNFSF6, Fc gamma RIII (CD16), Fc gamma RIIIA/CD16a, Fc gamma RIIIB/CD16b, ICOS, IL-18 R alpha/IL-1 R5, IL-23R, NKG2D/CD314, NKG2E, Occludin, TCR gamma/delta, TLR2, and TRAIL/TNFSF10.

6. The method of any one of claims 1-4, wherein the method results in neutralization of IL-2 binding and/or interaction with the IL-2 alpha-beta-gamma receptor.

7. The method of any one of claims 1-6, wherein the anti-IL-2 antibody, antibody format, or antigen binding portion thereof binds IL-2 in a manner that disrupts or blocks IL-2 interaction with the IL-2 alpha-beta-gamma receptor but does not disrupt or block IL-2 interaction with the beta-gamma complex.

8. The method of any one of the above claims, wherein the method reduces IL-2 signaling on a Treg cell.

9. The method of any one of the above claims, wherein the method causes selective proliferation and/or survival of CTLs or NK cells or Thelp cells.

10. The method of any one of the above claims, wherein the method selectively activates CTLs or NK cells or Thelp cells.

11. The method of any one of the above claims, wherein the method reduces the ratio of IL-2Ralpha-beta-gamma-mediated signaling to IL-2Rbeta-gamma-mediated signaling.

12. The method of any one of the above claims, wherein the method reduces the ratio of IL-2-modulated Treg cells to IL-2-modulated CTLs or NKs or Thelp cells.

13. The method of any one of the above claims, wherein the method increases IL-2-mediated STAT signaling on CTLs or NKs or Thelp cells.

14. The method of any one of the above claims, wherein the method decreases IL-2 mediated STAT signaling on Treg cells.

15. The method of any one of the above claims, wherein the method increases IL-2 sensitivity on CTLs or NKs or Thelp cells.

16. The method of any one of the above claims, wherein the method decreases IL-2 sensitivity on Treg cells.

17. The method of any one of the above claims, wherein the method causes the ratio of IL-2 sensitivity on Treg cells to CTLs and/or NK cells and/or Thelp cells to be about 1.

18. The method of any one of claims 1-17, wherein the method increases anti-tumor activity of IL-2 and decreases activation of Treg cells.

19. The method of claim 18, wherein the decrease of activation of Treg cells results in a decrease of T-cell suppression.

20. The method of any one of the above claims, wherein the construct does not comprise an exogenous wild-type IL-2 molecule.

21. The method of any one of the above claims, wherein the construct further comprises a targeting moiety comprising a recognition domain which specifically binds to a tissue-specific marker.

22. The method of claim 21, wherein the tissue-specific marker is selected from endothelial cell surface markers such as ACE, CD14, CD34, CDH5, ENG, ICAM2, MCAM, NOS3, PECAMI, PROCR, SELE, SELP, TEK, THBD, VCAMI, TEM1, TEM8, Clec14A, VWF; smooth muscle cell surface markers such as ACTA2, MYHIO, MYHI 1, MYH9, MYOCD; fibroblast (stromal) cell surface markers such as FAP, ALCAM, CD34, COLIAI, COL1A2, COL3A1, PH-4; epithelial cell surface markers such as CDID, K6IRS2, KRTIO, KRT13, KRT17, KRT18, KRT19, KRT4, KRT5, KRT8, MUCI, TACSTDI; neovasculature markers such as CD13, TFNA, Alpha-v beta-3 (aVB3), E-selectin; and adipocyte surface markers such as ADIPOQ, FABP4, and RETN.

23. The method of claim 21, wherein the tissue is the extracellular matrix (ECM).

24. The method of claim 23, wherein the ECM marker is selected from a tenascin, optionally selected from tenascin-C, tenascin-R, tenascin-X, and tenascin-W, a fibronectin, a fibrin, a laminin, and a nidogen/entactin.

25. The method of claim 22, wherein the tenascin is tenascin-CA1.

26. The method of any one of the above claims, wherein the antibody format is a single-chain antibody (scFv), a single-domain antibody, a recombinant heavy-chain-only antibody (VHH), a shark heavy-chain-only antibody (VNAR), a Fv, a Fab, a Fab′, or a F(ab′)₂.

27. The method of claim 26, wherein the antibody, antibody format, or antigen binding portion thereof is a scFv, or variant thereof.

28. The method of any one of the above claims, wherein the scFv, or variant thereof, comprises a VL-VH or VH-VL orientation.

29. The method of any one of the above claims, wherein the construct further comprises a Fc domain.

30. The method of claim 29, wherein the construct has an increased half-life as compared to a construct without a Fc domain.

31. The method of claim 29 or 30, wherein the Fc domain is a human IgG1 Fc backbone.

32. The method of any one of claims 29-31, wherein the Fc domain comprises effector mutations selected from one or more of L234A, L235A, P331G, and K322Q, or mutations corresponding thereto.

33. The method of any one of claims 29-32, wherein the Fc domain further comprises mutations selected from one or more of S354C and T366W, or mutations corresponding thereto.

34. The method of any one of claims 29-33, wherein the Fc domain further comprises mutations selected from one or more of Y349C, T366S, L368A, and Y407V, or mutations corresponding thereto.

35. The method of any one of the above claims, wherein the antibody, antibody format, or antigen binding portion thereof directed against an IL-2-responsive cell type marker, optionally a cytotoxic T lymphocyte (CTL) or natural killer cell (NK) or Thelp cell marker or gamma-delta T cell, is an anti-CD8 antibody, antibody format, or antigen binding portion thereof.

36. The method of claim 35, wherein the anti-CD8 antibody format is a single-domain antibody, a recombinant heavy-chain-only antibody (VHH), a single-chain antibody (scFv), a shark heavy-chain-only antibody (VNAR), a Fv, a Fab, a Fab′, or a F(ab′)₂.

37. The method of claim 36, wherein the anti-CD8 antibody format is a VHH or scFv.

38. The method of any one of the above claims, wherein the construct comprises a polypeptide having at least about 90% sequence similarity to SEQ ID NO: 12, without a leader sequence or a His₆ tag.

39. The method of any one of the above claims, wherein the subject is afflicted with cancer and/or wherein the method treats cancer in a subject.

40. The method of claim 39, wherein the cancer is selected form one or more of basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses; edema (e.g. that associated with brain tumors); and Meigs' syndrome.

41. The method of any one of the above claims, wherein the construct is a polypeptide.

42. The method of any one of the above claims, wherein the construct is a nucleic acid.

43. The method of claim 42, wherein the nucleic acid is or comprises deoxyribonucleic acid (DNA) and/or ribonucleic acid (RNA).

44. The method of claim 43, wherein the RNA is modified messenger RNA (mmRNA).

45. A method of reducing, mitigating, or eliminating endogenous IL-2 signaling and/or binding to the IL-2 alpha-beta-gamma receptor in a subject, the method comprising administering to the subject a construct comprising
(i) an anti-IL-2 antibody, antibody format, or antigen binding portion thereof, which binds IL-2 in a manner that disrupts or blocks IL-2 interaction with the IL-2 alpha-beta-gamma receptor, and
(ii) one or more antibody, antibody format, or antigen binding portion thereof directed against a Treg cell marker,
wherein the anti-IL-2 antibody, antibody format, or antigen binding portion thereof and the one or more antibody, antibody format, or antigen binding portion thereof directed against a Treg cell marker are linked.

46. A method of redirecting endogenous IL-2 signaling from the IL-2 alpha-beta-gamma receptor to the IL-2 beta-gamma receptor on an antigen-expressing cell in a subject, the method comprising administering to the subject a construct comprising:
(i) an anti-IL-2 antibody, antibody format, or antigen binding portion thereof, which binds IL-2 in a manner that disrupts or blocks IL-2 interaction with the IL-2 alpha-beta-gamma receptor, and
(ii) one or more antibody, antibody format, or antigen binding portion thereof directed against a Treg cell marker,
wherein the anti-IL-2 antibody, antibody format, or antigen binding portion thereof and the one or more antibody, antibody format, or antigen binding portion thereof directed against a Treg cell marker are linked.

47. The method of claim 45 or 46, wherein the anti-IL-2 antibody, antibody format, or antigen binding portion thereof binds IL-2 in a manner that disrupts or blocks IL-2 interaction with the IL-2 alpha-beta-gamma receptor but does not disrupt or block IL-2 interaction with the beta-gamma complex.

48. The method of claim 45 or 46, wherein the endogenous IL-2 is redirected from activated CTL or NK or Thelp cells to Treg cells.

49. The method of any one of claims 45-48, wherein the Treg cell marker is selected from CTLA-4, CD25, GITR, CD127, LAG-3, PD-1, and CCR8.

50. The method of any one of claims 45-49, wherein the method results in neutralization of IL-2 binding and/or interaction with the IL-2 alpha-beta-gamma receptor.

51. The method of any one of claims 45-50, wherein the method reduces IL-2 signaling on an activated CTL or NK or Thelp cell.

52. The method of any one of claims 45-50, wherein the method causes selective proliferation and/or survival of Treg cells.

53. The method of any one of claims 45-52, wherein the method selectively activates Treg cells.

54. The method of any one of claims 45-53, wherein the method reduces the ratio of IL-2R alpha-beta-gamma-mediated signaling to IL-2R beta-gamma-mediated signaling.

55. The method of any one of claims 45-54, wherein the method reduces the ratio of IL-2-modulated CTL cells to IL-2-modulated Treg cells.

56. The method of any one of claims 45-55, wherein the method increases IL-2-mediated STAT signaling in Treg cells.

57. The method of any one of claims 45-56, wherein the method decreases IL-2 mediated STAT signaling in CTL or NK or Thelp cells.

58. The method of any one of claims 45-57, wherein the method increases IL-2 sensitivity in Treg cells.

59. The method of any one of claims 45-58, wherein the method decreases IL-2 sensitivity on CTL or NK or Thelp cells.

60. The method of any one of claims 45-59, wherein the method causes the ratio of IL-2 sensitivity on CTL or NK or Thelp cells to Treg cells to be about 1.

61. The method of any one of claims 45-60, wherein the decrease of activation of CTL or NK or Thelp cells results in a dampened immune response.

62. The method of any one of claims 45-61, wherein the construct does not comprise an exogenous wild-type IL-2 molecule.

63. The method of any one of claims 45-62, wherein the construct further comprises a targeting moiety comprising a recognition domain which specifically binds to a tissue-specific marker.

64. The method of claim 63, wherein the tissue specific-marker is selected from endothelial cell surface markers such as ACE, CD14, CD34, CDH5, ENG, ICAM2, MCAM, NOS3, PECAMI, PROCR, SELE, SELP, TEK, THBD, VCAMI, VWF; smooth muscle cell surface markers such as ACTA2, MYHIO, MYHI 1, MYH9, MYOCD; fibroblast (stromal) cell surface markers such as FAP, ALCAM, CD34, COLIAI, COL1A2, COL3A1, PH-4; epithelial cell surface markers such as CDID, K6IRS2, KRTIO, KRT13, KRT17, KRT18, KRT19, KRT4, KRT5, KRT8, MUCI, TACSTDI; neovasculature markers such as CD13, TFNA, Alpha-v beta-3 (aVB3), E-selectin; and adipocyte surface markers such as ADIPOQ, FABP4, and RETN.

65. The method of claim 63, wherein the tissue is the extracellular matrix (ECM).

66. The method of claim 65, wherein the ECM marker is selected from a tenascin, optionally selected from tenascin-C, tenascin-R, tenascin-X, and tenascin-W, a fibronectin, a fibrin, a laminin, and a nidogen/entactin.

67. The method of claim 66, wherein the tenascin is tenascin-CA1.

68. The method of any one of claims 45-67, wherein the antibody format is a single-chain antibody (scFv), a single-domain antibody, a recombinant heavy-chain-only antibody (VHH), a shark heavy-chain-only antibody (VNAR), a Fv, a Fab, a Fab′, or a F(ab′)₂.

69. The method of claim 68, wherein the antibody, antibody format, or antigen binding portion thereof is a scFv, or variant thereof.

70. The method of any one of claims 45-69, wherein the scFv, or variant thereof, comprises a VL-VH or VH-VL orientation.

71. The method of any one of claims 45-70, wherein the construct further comprises a Fc domain.

72. The method of claim 71, wherein construct has an increased half-life as compared to a construct without a Fc domain.

73. The method of claim 71 or 72, wherein the Fc domain is a human IgG1 Fc backbone.

74. The method of any one of claims 71-73, wherein the Fc domain comprises effector mutations selected from one or more of L234A, L235A, P331G, and K322Q, or mutations corresponding thereto.

75. The method of any one of claims 71-74, wherein the Fc domain further comprises mutations selected from one or more of S354C and T366W, or mutations corresponding thereto.

76. The method of any one of claims 71-75, wherein the Fc domain further comprises mutations selected from one or more of Y349C, T366S, L368A, and Y407V, or mutations corresponding thereto.

77. The method of any one of claism 45-76, wherein the antibody, antibody format, or antigen binding portion thereof directed against a Treg cell marker selected from an anti-CTLA-4, anti-CD25, anti-GITR, anti-CD127, anti-LAG-3, anti-PD-1, or anti-CCR8 antibody, antibody format, or antigen binding portion thereof.

78. The method of claim 77, wherein the an anti-CTLA-4, anti-CD25, anti-GITR, anti-CD127, anti-LAG-3, anti-PD-1, or anti-CCR8 antibody format is a single-domain antibody scFv, a recombinant heavy-chain-only antibody (VHH), a single-chain antibody (scFv), a shark heavy-chain-only antibody (VNAR), a Fv, a Fab, a Fab′, or a F(ab′)₂.

79. The method of claim 78, wherein the anti-CTLA-4 antibody format is a VHH or scFv.

80. The method of any one of claims 45-79, wherein the subject is afflicted with an auto-immune disease or disorder or an inflammation-related disorder.

81. The method of claim 80, wherein the auto-immune disease or disorder is selected form one or more of multiple sclerosis, celiac disease, rheumatoid arthritis, systemic lupus erythematosus, Sjogren's syndrome, polymyalgia rheumatica, ankylosing spondylitis, type 1 diabetes, vasculitis, temporal arteritis, Graves disease, dermatomyositis, Addison disease, Hashimoto thyroiditis, Myasthenia gravis, and pernicious anemia.

82. The method of claim 80, wherein the inflammation-related disorder is selected from one or more of inflammation, acute inflammation, chronic inflammation, respiratory disease, atherosclerosis, restenosis, asthma, allergic rhinitis, atopic dermatitis, septic shock, rheumatoid arthritis, inflammatory bowel disease, inflammatory pelvic disease, pain, ocular inflammatory disease, celiac disease, Leigh Syndrome, Glycerol Kinase Deficiency, Familial eosinophilia (FE), autosomal recessive spastic ataxia, laryngeal inflammatory disease; Tuberculosis, Chronic cholecystitis, Bronchiectasis, Silicosis and other pneumoconiosis.

83. The method of any one of claims 45-82, wherein the construct is a polypeptide.

84. The method of any one of claims 45-82, wherein the construct is a nucleic acid.

85. The method of claim 84, wherein the nucleic acid is or comprises deoxyribonucleic acid (DNA) and/or ribonucleic acid (RNA).

86. The method of claim 85, wherein the RNA is modified messenger RNA (mmRNA).

87. A composition comprising a polypeptide having at least about 90%, at least about 93%, at least about 95%, at least about 96%, at least about 97%, at least about 98%, at least about 99%, or at least about 100% sequence similarity to one of SEQ ID NO: 1-12, with or without a leader sequence and/or with or without a His₆ tag.

88. A nucleic acid encoding the composition comprising the polypeptide of claim 87.

89. The nucleic acid of claim 88, wherein the nucleic acid is or comprises deoxyribonucleic acid (DNA) and/or ribonucleic acid (RNA).

90. The nucleic acid of claim 89, wherein the RNA is modified messenger RNA (mmRNA).


SEQUENCE LISTING (53 sequences)

SEQ ID NO: 1 - protein, 527 aa, synthetic construct
  REGION 1..527: note = Synthetic polypeptide.
MEFGLSWLFLVAILKGVQCQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSGGGGSGGGGSGGGGSDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKGGGGSGGGGSGGGGSEVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSGGGGSGGGGSGGGGSDVQITQSPSSLSASVGDRVTITCRTSRSISQYLAWYQEKPGKTNKLLIYSGSTLQSGIPSRFSGSGSGTDFTLTISSLQPEDFATYYCQQVNEFPPTFGQGTKVEIKHHHHHH

SEQ ID NO: 2 - protein, 527 aa, synthetic construct
  REGION 1..527: note = Synthetic polypeptide.
MEFGLSWLFLVAILKGVQCDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKGGGGSGGGGSGGGGSQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSGGGGSGGGGSGGGGSEVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSGGGGSGGGGSGGGGSDVQITQSPSSLSASVGDRVTITCRTSRSISQYLAWYQEKPGKTNKLLIYSGSTLQSGIPSRFSGSGSGTDFTLTISSLQPEDFATYYCQQVNEFPPTFGQGTKVEIKHHHHHH

SEQ ID NO: 3 - protein, 523 aa, synthetic construct
  REGION 1..523: note = Synthetic polypeptide.
MEFGLSWLFLVAILKGVQCEVQLQQSGAELARPGASVKLSCKASGYTFTTYWIQWVKQRPGQGLEWIGAIYPGDGDTRYIQNFKGKATLTADKSSSTAYMQLSSLASEDSAVYYCARSLATRGFYAMDYWGQGTSVTVSSGGGGSGGGGSGGGGSDIVMTQSPASLSMAIGEKVTIRCITSTDIDDDMNWYQQKPGEPPKLLISEGNTLRPGVPSRFSSSGYGTDFVFTIENMLSEDVADYYCLQSDNLPYTFGGGTKLEIKGGGGSGGGGSGGGGSEVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSGGGGSGGGGSGGGGSDVQITQSPSSLSASVGDRVTITCRTSRSISQYLAWYQEKPGKTNKLLIYSGSTLQSGIPSRFSGSGSGTDFTLTISSLQPEDFATYYCQQVNEFPPTFGQGTKVEIKHHHHHH

SEQ ID NO: 4 - protein, 523 aa, synthetic construct
  REGION 1..523: note = Synthetic polypeptide.
MEFGLSWLFLVAILKGVQCDIVMTQSPASLSMAIGEKVTIRCITSTDIDDDMNWYQQKPGEPPKLLISEGNTLRPGVPSRFSSSGYGTDFVFTIENMLSEDVADYYCLQSDNLPYTFGGGTKLEIKGGGGSGGGGSGGGGSEVQLQQSGAELARPGASVKLSCKASGYTFTTYWIQWVKQRPGQGLEWIGAIYPGDGDTRYIQNFKGKATLTADKSSSTAYMQLSSLASEDSAVYYCARSLATRGFYAMDYWGQGTSVTVSSGGGGSGGGGSGGGGSEVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSGGGGSGGGGSGGGGSDVQITQSPSSLSASVGDRVTITCRTSRSISQYLAWYQEKPGKTNKLLIYSGSTLQSGIPSRFSGSGSGTDFTLTISSLQPEDFATYYCQQVNEFPPTFGQGTKVEIKHHHHHH

SEQ ID NO: 5 - protein, 272 aa, synthetic construct
  REGION 1..272: note = Synthetic polypeptide.
MEFGLSWLFLVAILKGVQCQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSGGGGSGGGGSGGGGSDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKHHHHHH

SEQ ID NO: 6 - protein, 272 aa, synthetic construct
  REGION 1..272: note = Synthetic polypeptide.
MEFGLSWLFLVAILKGVQCDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKGGGGSGGGGSGGGGSQVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSHHHHHH

SEQ ID NO: 7 - protein, 268 aa, synthetic construct
  REGION 1..268: note = Synthetic polypeptide.
MEFGLSWLFLVAILKGVQCEVQLQQSGAELARPGASVKLSCKASGYTFTTYWIQWVKQRPGQGLEWIGAIYPGDGDTRYIQNFKGKATLTADKSSSTAYMQLSSLASEDSAVYYCARSLATRGFYAMDYWGQGTSVTVSSGGGGSGGGGSGGGGSDIVMTQSPASLSMAIGEKVTIRCITSTDIDDDMNWYQQKPGEPPKLLISEGNTLRPGVPSRFSSSGYGTDFVFTIENMLSEDVADYYCLQSDNLPYTFGGGTKLEIKHHHHHH

SEQ ID NO: 8 - protein, 268 aa, synthetic construct
  REGION 1..268: note = Synthetic polypeptide.
MEFGLSWLFLVAILKGVQCDIVMTQSPASLSMAIGEKVTIRCITSTDIDDDMNWYQQKPGEPPKLLISEGNTLRPGVPSRFSSSGYGTDFVFTIENMLSEDVADYYCLQSDNLPYTFGGGTKLEIKGGGGSGGGGSGGGGSEVQLQQSGAELARPGASVKLSCKASGYTFTTYWIQWVKQRPGQGLEWIGAIYPGDGDTRYIQNFKGKATLTADKSSSTAYMQLSSLASEDSAVYYCARSLATRGFYAMDYWGQGTSVTVSSHHHHHH

SEQ ID NO: 9 - protein, 265 aa, synthetic construct
  REGION 1..265: note = Synthetic polypeptide.
MEFGLSWLFLVAILKGVQCEVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSGGGGSGGGGSGGGGSDVQITQSPSSLSASVGDRVTITCRTSRSISQYLAWYQEKPGKTNKLLIYSGSTLQSGIPSRFSGSGSGTDFTLTISSLQPEDFATYYCQQVNEFPPTFGQGTKVEIKHHHHHH

SEQ ID NO: 10 - protein, 504 aa, synthetic construct
  REGION 1..504: note = Synthetic polypeptide.
MEFGLSWLFLVAILKGVQCDIVMTQSPASLSMAIGEKVTIRCITSTDIDDDMNWYQQKPGEPPKLLISEGNTLRPGVPSRFSSSGYGTDFVFTIENMLSEDVADYYCLQSDNLPYTFGGGTKLEIKGGGGSGGGGSGGGGSEVQLQQSGAELARPGASVKLSCKASGYTFTTYWIQWVKQRPGQGLEWIGAIYPGDGDTRYIQNFKGKATLTADKSSSTAYMQLSSLASEDSAVYYCARSLATRGFYAMDYWGQGTSVTVSSGGSGGSGGSGGSGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 11 - protein, 519 aa, synthetic construct
  REGION 1..519: note = Synthetic polypeptide.
MDMRVPAQLLGLLLLWLRGARCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAGIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGSGGSGGSGGSGGSGGSGGSGGSEVQLVQSGAEVKKPGASVKVSCKASGFNIKDTYIHWVRQAPGQGLEWIGRIDPANDNTLYASKFQGRATITADTSTSTAYLELSSLRSEDTAVYYCGRGYGYYVFDHWGQGTLVTVSSGGGGSGGGGSGGGGSDVQITQSPSSLSASVGDRVTITCRTSRSISQYLAWYQEKPGKTNKLLIYSGSTLQSGIPSRFSGSGSGTDFTLTISSLQPEDFATYYCQQVNEFPPTFGQGTKVEIK

SEQ ID NO: 12 - protein, 403 aa, synthetic construct
  REGION 1..403: note = Synthetic polypeptide.
MEFGLSWLFLVAILKGVQCDVQLQESGGGLVQPGGSLRLSCAASGSIFSINVMGWYRQTPGKERELVAKITNFGITSYADSAQGRFTISRGNAKNTVYLQMNSLKPEDTAVYYCNLDTTGWGPPPYQYWGQGTQVTVSSGGGGSGGGGSGGGGSDIVMTQSPASLSMAIGEKVTIRCITSTDIDDDMNWYQQKPGEPPKLLISEGNTLRPGVPSRFSSSGYGTDFVFTIENMLSEDVADYYCLQSDNLPYTFGGGTKLEIKGGGGSGGGGSGGGGSEVQLQQSGAELARPGASVKLSCKASGYTFTTYWIQWVKQRPGQGLEWIGAIYPGDGDTRYIQNFKGKATLTADKSSSTAYMQLSSLASEDSAVYYCARSLATRGFYAMDYWGQGTSVTVSSHHHHHH

SEQ ID NO: 13 - protein, 5 aa, synthetic construct
  REGION 1..5: note = Synthetic polypeptide.
GGGGS

SEQ ID NO: 14 - protein, 10 aa, synthetic construct
  REGION 1..10: note = Synthetic polypeptide.
GGGGSGGGGS

SEQ ID NO: 15 - protein, 15 aa, synthetic construct
  REGION 1..15: note = Synthetic polypeptide.
GGGGSGGGGSGGGGS

SEQ ID NO: 16 - protein, 20 aa, synthetic construct
  REGION 1..20: note = Synthetic polypeptide.
GGGGSGGGGSGGGGSGGGGS

SEQ ID NO: 17 - protein, 25 aa, synthetic construct
  REGION 1..25: note = Synthetic polypeptide.
GGGGSGGGGSGGGGSGGGGSGGGGS

SEQ ID NO: 18 - protein, 30 aa, synthetic construct
  REGION 1..30: note = Synthetic polypeptide.
GGGGSGGGGSGGGGSGGGGSGGGGSGGGGS

SEQ ID NO: 19 - protein, 35 aa, synthetic construct
  REGION 1..35: note = Synthetic polypeptide.
GGGGSGGGGSGGGGSGGGGSGGGGSGGGGSGGGGS

SEQ ID NO: 20 - protein, 40 aa, synthetic construct
  REGION 1..40: note = Synthetic polypeptide.
GGGGSGGGGSGGGGSGGGGSGGGGSGGGGSGGGGSGGGGS

SEQ ID NO: 21 - protein, 4 aa, synthetic construct
  REGION 1..4: note = Synthetic polypeptide.
GGGS

SEQ ID NO: 22 - protein, 8 aa, synthetic construct
  REGION 1..8: note = Synthetic polypeptide.
GGGSGGGS

SEQ ID NO: 23 - protein, 12 aa, synthetic construct
  REGION 1..12: note = Synthetic polypeptide.
GGGSGGGSGGGS

SEQ ID NO: 24 - protein, 16 aa, synthetic construct
  REGION 1..16: note = Synthetic polypeptide.
GGGSGGGSGGGSGGGS

SEQ ID NO: 25 - protein, 20 aa, synthetic construct
  REGION 1..20: note = Synthetic polypeptide.
GGGSGGGSGGGSGGGSGGGS

SEQ ID NO: 26 - protein, 24 aa, synthetic construct
  REGION 1..24: note = Synthetic polypeptide.
GGGSGGGSGGGSGGGSGGGSGGGS

SEQ ID NO: 27 - protein, 28 aa, synthetic construct
  REGION 1..28: note = Synthetic polypeptide.
GGGSGGGSGGGSGGGSGGGSGGGSGGGS

SEQ ID NO: 28 - protein, 32 aa, synthetic construct
  REGION 1..32: note = Synthetic polypeptide.
GGGSGGGSGGGSGGGSGGGSGGGSGGGSGGGS

SEQ ID NO: 29 - protein, 16 aa, synthetic construct
  REGION 1..16: note = Synthetic polypeptide.
GGSGGSGGGGSGGGGS

SEQ ID NO: 30 - protein, 5 aa, synthetic construct
  REGION 1..5: note = Synthetic polypeptide.
GGGGS

SEQ ID NO: 31 - protein, 5 aa, synthetic construct
  REGION 1..5: note = Synthetic polypeptide.
GGGGS

SEQ ID NO: 32 - protein, 10 aa, synthetic construct
  REGION 1..10: note = Synthetic polypeptide.
GGGGSGGGGS

SEQ ID NO: 33 - protein, 15 aa, synthetic construct
  REGION 1..15: note = Synthetic polypeptide.
GGGGSGGGGSGGGGS

SEQ ID NO: 34 - protein, 20 aa, synthetic construct
  REGION 1..20: note = Synthetic polypeptide.
GGGGSGGGGSGGGGSGGGGS

SEQ ID NO: 35 - protein, 8 aa, synthetic construct
  REGION 1..8: note = Synthetic polypeptide.
GGGGGGGG

SEQ ID NO: 36 - protein, 6 aa, synthetic construct
  REGION 1..6: note = Synthetic polypeptide.
GGGGGG

SEQ ID NO: 37 - protein, 5 aa, synthetic construct
  REGION 1..5: note = Synthetic polypeptide.
EAAAK

SEQ ID NO: 38 - protein, 10 aa, synthetic construct
  REGION 1..10: note = Synthetic polypeptide.
EAAAKEAAAK

SEQ ID NO: 39 - protein, 15 aa, synthetic construct
  REGION 1..15: note = Synthetic polypeptide.
EAAAKEAAAKEAAAK

SEQ ID NO: 40 - protein, 12 aa, synthetic construct
  REGION 1..12: note = Synthetic polypeptide.
AEAAAKEAAAKA

SEQ ID NO: 41 - protein, 17 aa, synthetic construct
  REGION 1..17: note = Synthetic polypeptide.
AEAAAKEAAAKEAAAKA

SEQ ID NO: 42 - protein, 22 aa, synthetic construct
  REGION 1..22: note = Synthetic polypeptide.
AEAAAKEAAAKEAAAKEAAAKA

SEQ ID NO: 43 - protein, 27 aa, synthetic construct
  REGION 1..27: note = Synthetic polypeptide.
AEAAAKEAAAKEAAAKEAAAKEAAAKA

SEQ ID NO: 44 - protein, 12 aa, synthetic construct
  REGION 1..12: note = Synthetic polypeptide.
AEAAAKEAAAKA

SEQ ID NO: 45 - protein, 46 aa, synthetic construct
  REGION 1..46: note = Synthetic polypeptide.
AEAAAKEAAAKEAAAKEAAAKALEAEAAAKEAAAKEAAAKEAAAKA

SEQ ID NO: 46 - protein, 5 aa, synthetic construct
  REGION 1..5: note = Synthetic polypeptide.
PAPAP

SEQ ID NO: 47 - protein, 18 aa, synthetic construct
  REGION 1..18: note = Synthetic polypeptide.
KESGSVSSEQLAQFRSLD

SEQ ID NO: 48 - protein, 14 aa, synthetic construct
  REGION 1..14: note = Synthetic polypeptide.
EGKSSGSGSESKST

SEQ ID NO: 49 - protein, 12 aa, synthetic construct
  REGION 1..12: note = Synthetic polypeptide.
GSAGSAAGSGEF

SEQ ID NO: 50 - protein, 5 aa, synthetic construct
  REGION 1..5: note = Synthetic polypeptide.
GGGSE

SEQ ID NO: 51 - protein, 5 aa, synthetic construct
  REGION 1..5: note = Synthetic polypeptide.
GSESG

SEQ ID NO: 52 - protein, 5 aa, synthetic construct
  REGION 1..5: note = Synthetic polypeptide.
GSEGS

SEQ ID NO: 53 - protein, 35 aa, synthetic construct
  REGION 1..35: note = Synthetic polypeptide.
GEGGSGEGSSGEGSSSEGGGSEGGGSEGGGSEGGS